SEQUENCE LISTING (8 sequences)

SEQ ID NO: 1 - DNA, 20 bp, artificial sequence
  Forward primer for amplifying sphingosine-1- phosphate (S1P) receptor S1P1
gcgctcagagacttcgtctt

SEQ ID NO: 2 - DNA, 20 bp, artificial sequence
  Reverse primer for amplifying sphingosine-1- phosphate (S1P) receptor S1P1
accagctcactcgcaaagtt

SEQ ID NO: 3 - DNA, 20 bp, artificial sequence
  Forward primer for amplifying sphingosine-1- phosphate (S1P) receptor S1P3
ccttgcagaacgagagccta

SEQ ID NO: 4 - DNA, 20 bp, artificial sequence
  Reverse primer for amplifying sphingosine-1- phosphate (S1P) receptor S1P3
ttcccggagagtgtcatttc

SEQ ID NO: 5 - DNA, 20 bp, artificial sequence
  Primer for amplifying GAPDH
ctcatgaccacagtccatgc

SEQ ID NO: 6 - protein, 184 aa, Homo sapiens [sequence omitted]

SEQ ID NO: 7 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 8 - protein, 175 aa, Homo sapiens
MGARRLRVRSQRSRDSSVPTQCNQTECFDPLVRNCVSCELFHTPDTGHTSSLEPGTALQPQEGSALRPDVALLVGAPALLGLILALTLVGLVSLVSWRWRQQLRTASPDTSEGVQQESLENVFVPSSETPHASAPTWPPLKEDADSALPRHSVPVPATELGSTELVTTKTAGPEQ

(19) United States
(12) Patent Application Publication
(10) Pub. No.: US 2009/0226440 A1
(43) Pub. Date: Sep. 10, 2009

(54) Prophylactic and/or Therapeutic Method for Treatment of Autoimmune Disease

(76) Inventor: Shane Grey, New South Wales (AU)

Correspondence Address: COOPER & DUNHAM, LLP, 30 Rockefeller Plaza, 20th Floor, NEW YORK, NY, 10112, US

(21) Appl. No.: 11/990,428

(22) PCT Filed: Aug. 14, 2006
(86) PCT No.: PCT/AU2006/001163

§ 371 Date: Sep. 22, 2008

Related U.S. Application Data
(60) Provisional application No. 60/707,803, filed on Aug. 12, 2005.

(51) Int. Cl. A61K 39/395

(52) U.S. Cl. 424/134.1; 424/133.1

(57) ABSTRACT

The present invention provides therapeutic and/or prophylactic method comprising administering to a subject an amount of a composition sufficient to reduce or deplete antibody producing cells and/or prevent expansion of said cells in a tissue or organ of a subject suffering from T cell mediated autoimmune disease e.g., type 1 diabetes, or at risk of suffering from said disease, preferably wherein the composition is administered immediately prior to or concomitant with an autoimmune response. The present invention also provides the use of said composition in the manufacture of a medicament for the treatment and/or prevention of T cell mediated autoimmune disease. The present invention also provides said composition for use in the treatment and/or prevention of T cell mediated autoimmune disease.

### FIELD OF THE INVENTION

The present invention relates to a prophylactic and/or therapeutic method for treatment of autoimmune disease, preferably T cell-mediated autoimmune diseases such as, for example, type 1 diabetes. The invention also relates to the use of compositions of matter that reduce or deplete antibody producing cells (B cells) and/or prevent expansion of said cells for treatment.

### BACKGROUND OF THE INVENTION

### General

This specification contains nucleotide and amino acid sequence information prepared using PatentIn Version 3.3, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g., SEQ ID NO: 1 refers to the sequence in the sequence listing designated as <400>1).

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise. The features of each and every embodiment of the invention described herein for prophylactic and/or therapeutic treatment of T cell mediated autoimmune disease are to be applied mutatis mutandis to any use or medical indication of a composition for the prophylactic and/or therapeutic treatment of T cell mediated autoimmune disease. Similarly, the features of each and every embodiment of the invention described herein for prophylactic and/or therapeutic treatment of type 1 diabetes are to be applied mutatis mutandis to any use or medical indication of a composition for the prophylactic and/or therapeutic treatment of type 1 diabetes.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
1. Sambrook, Fritsch and Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Animal Cell Culture: Practical Approach, Third Edition (John R. W. Masters, ed., 2000), ISBN 0199637970, whole of text;
4. Handbook of Experimental Immunology, Vols. I-IV (D. M. Weir and C. C. Blackwell, eds., 1986, Blackwell Scientific Publications).

### DESCRIPTION OF THE RELATED ART

Diabetes Mellitus is one of the most common chronic endocrine disorders across all age groups and populations. This disease is characterized by high levels of blood glucose resulting from defects in insulin production and/or insulin action.

The hormone, insulin is essential in the metabolism of carbohydrates, fat, and protein. Insulin reduces blood glucose levels by allowing glucose to enter muscle cells and fat cells and by stimulating the conversion of glucose to glycogen (glycogenesis) as a carbohydrate store. Insulin also inhibits the release of stored glucose from liver glycogen (glycogenolysis) and slows the breakdown of fat to triglycerides, free fatty acids, and ketones. Additionally, insulin slows the breakdown of protein for glucose production (gluconeogenesis).

Generally, diabetes is classed as one of two types. Type 1 diabetes (or insulin-dependent diabetes mellitus; IDDM) is caused by the absence, destruction, or loss of pancreatic β-cells resulting in an absolute deficiency of insulin. Type 2 diabetes (non-insulin dependent diabetes; NIDDM) is a heterogeneous disorder that is characterized by insulin resistance.

### Type 1 Diabetes

The overall incidence of type 1 diabetes is approximately 15 cases per 100,000 individuals in the US alone. Approximately, 5 to 15 percent of all cases of diabetes are type 1 diabetes cases in the US, with physicians diagnosing about 10,000 new cases every year. Internationally, the incidence of type 1 diabetes varies from about 0.61 cases per 100,000 individuals in China to about 34.5 cases per 100,000 in Sardinia, and more than 40 cases per 100,000 in Finland. Many countries also report that the incidence rate of type 1 diabetes has doubled over the last 20 years.

The acute clinical onset of type 1 diabetes is characterized by symptoms, such as, for example, hyperglycemia (polyuria, polydipsia, weight loss, or blurred vision, alone or in combination), followed days or weeks later by ketoacidosis. Generally, the acute onset of the disease is considered to be preceded by a long, asymptomatic preclinical period, during which the insulin-secreting β-cells are progressively destroyed by the subjects own immune system.

In healthy individuals, the pancreas normally contains 1 to 1.5 million islets; and approximately 80 percent of islet cells are insulin-producing β-cells. The symptoms of clinical diabetes appear when fewer than 10 percent of those β-cells remain.

The progressive destruction of the body's ability to regulate glucose metabolism is believed to be caused by insulitis, or lymphocytic infiltration of the pancreatic islets, with concomitant changes in T cell subpopulations, such as increased suppressor-inducer T cells and decreased helper-inducer T cells. Furthermore, antibodies are produced against several auto-antigens, such as, for example, insulin, GAD65 and IA-2.

The mismatch between insulin supply and demand caused by the loss of pancreatic β-islet cells leads to abnormal glucose, lipid and protein metabolism. Insulin deficiency may lead to hyperglycemia and hyperglycemic dehydration, elevated levels of free fatty acids, elevated serum ketone levels, increased levels of triglycerides, increased levels of very low density lipoproteins (VLDLs), increased levels of branched chain amino acids, a decrease in protein synthesis, and ketoacidosis. A subject with type 1 diabetes is likely to suffer from any one or more of a variety of vascular and neurologic complications. For example, type 1 diabetes patients are two times more likely than non-diabetics to have a heart attack; they are five times more likely to suffer from gangrene; seventeen times more likely to have complete renal failure, and twenty-five times more likely to lose their eyesight.

### Treatment/Prophylaxis of Type 1 Diabetes

Currently, type 1 diabetes is treated by administration of exogenous insulin, exercise and dietary management. These forms of therapy do not correct the damage to the pancreas (i.e., replace the destroyed β-islet cells), but rather replace growth factors produced by the β-islet cells or attempt to avoid the requirement for these factors.

Most subjects suffering from type 1 diabetes requires some form of insulin therapy. At this time, such therapy generally requires the subject monitoring blood glucose and/or insulin levels and injecting recombinant or purified insulin when required. New forms of insulin are also being developed to enable nasal or oral administration. However, this form of therapy requires continual monitoring by the subject and insulin administration at least once a day for the life of the subject. Should the subject neglect to administer insulin or administer too much insulin there is a risk of the development of, for example, hyperglycemia, hypoglycemia or ketoacidosis.

Other compounds currently used for the treatment of type 1 diabetes include for example, sulfonylurea, biguanide, α-glucosidase inhibitor or thiazolidinedione. However, each of these compounds also suffer from significant disadvantages. For example, sulfonylurea causes hypoglycemia and hyperinsulinemia; biguanide causes lactic acidosis; α-glucosidase inhibitor causes gastro-intestinal side-effects; and thiazolidinedione has a long-onset of action, is associated with weight gain and requires frequent liver function testing.

Several studies have also been performed to determine suitable prophylactic compounds and/or treatments to prevent the onset of type 1 diabetes. To date, few of these treatments have been tested in humans. Rather, rodent models are used in pre-clinical studies to determine the efficacy of such a treatment. In particular, the non-obese diabetic (NOD) mouse model and the biobreeding (BB) rat model of type 1 diabetes are used to study potential prophylactic compounds.

As type 1 diabetes is considered to be an autoimmune disease, the studies performed to date have focused on the suppression of such an immune response to thereby prevent disease onset. Generally, these studies have involved administering a protein or peptide against which a diabetic subject raises an immune response to a subject at risk of developing diabetes to induce production of a tolerance response.

For example, using the NOD mouse, Zhang et al., Proc. Natl. Acad. Sci. USA, 88: 10252-10256, 1991 showed that oral administration of insulin to mice suffering from insulitis delays the onset of diabetes. Furthermore, the authors showed that a greater degree of protection was attained using younger animals at the time of therapy. The disadvantage of this form of treatment is that it requires ongoing administration of insulin to ensure an effect. Furthermore, the treatment was not sufficient to prevent disease onset, merely delay the age at which the disease symptoms were observable.

Similar studies have been performed using fragments of insulin or the diabetes auto antigen GAD65. Again, while these forms of treatment were successful in slowing the onset of disease, the treated animals still developed diabetes Ramiya et al., J. Autoimmun. 9: 349-356, 1999.

Accordingly, it is clear that there is a need in the art for a method to prevent the onset of type 1 diabetes or to reduce type 1 diabetes disease progression. Preferably, such a method prevents the loss of sufficient β-islet cells in a subject to require ongoing treatment with exogenous insulin.

### SUMMARY OF INVENTION

In work leading up to the present invention the inventors sought to develop a better understanding of the immune response that is responsible for the destruction of pancreatic β-islet cells during the development of diabetes. The inventors used the well-established NOD mouse as a model of type 1 diabetes in humans.

The studies performed by the inventors showed that while NOD mice were lymphopenic, they had normal ratios of B:T cells compared to non-diabetic mouse strains. However, the NOD mice had significantly increased numbers of marginal zone B cells (MZB cells) compared to non-diabetic controls. Furthermore, B cells from NOD mice were hyperactive, with antibody responses to T-dependent antigens being higher compared to the response by B cells from non-diabetic controls.

The inventors also found that B cells increase in number or are enhanced in spleen and/or whole blood and/or pancreatic lymph node tissue (e.g., by expansion and/or reduced depletion and/or reduced turnover) immediately prior to the onset of clinically detectable disease in NOD mice. Together, these results indicate a role for B cells in disease onset and/or progression. The inventors have also found that, by depleting B cells and/or preventing B cell expansion in NOD mice (an accepted model of diabetes) using a compound that prevents this change in B cell profile, the mice did not develop diabetes. Accordingly, these studies form the basis of novel prophylactic and/or therapeutic method(s) for the treatment of type 1 diabetes.

Accordingly, the present invention provides a therapeutic and/or prophylactic method comprising administering to a subject an amount of a composition sufficient to reduce or deplete antibody producing cells and/or prevent expansion of said cells in a tissue or organ of a subject suffering from a T cell mediated autoimmune disease or at risk of suffering from said disease. For example, the composition is administered immediately prior to or concomitant with an autoimmune response such as indicated by expansion of a population of T cells and/or B cells and/or by the production of autoantibodies (e.g., expansion of cytotoxic T cells against pancreatic β-islet cells and/or autoantibodies against one or more pancreatic β-islet cell markers in the onset or progression of type 1 diabetes) and/or by an increase in serum glucose levels.

A disease can be classified as autoimmune if there is an adaptive immune response to a self-antigen causing the observed pathology, involving autoantibodies and/or autoreactive T cells. As used herein, a “T cell-mediated autoimmune disease” is an autoimmune disease directed to one or more affected organs or tissues and for which there is an adaptive immune response to a self-antigen comprising the presence or an accumulation of autoreactive T cells in affected organ or tissue, and wherein the immune response in involved the immunopathology of the disease as demonstrated in humans and/or animal models of the disease and not merely coincident with the disease such that (i) adoptive transfer of autoreactive T cells or immunization with autoantigen transfers/induces the disease to healthy animals and (ii) elimination or suppression of the autoimmune response prevents disease progression and/or prevents or ameliorates clinical manifestation of the disease. For those diseases where immunopathology is theoretical and not supported by animal models or clinical data, immunopathology of a T cell-mediated autoimmune disease should preferably not be explained by the action of autoantibodies.

Examples of T cell-mediated autoimmune disease include but are not limited to type-1 diabetes (T1D) and complications arising therefrom e.g., graft versus host disease including rejection of β-islet cell graft, multiple sclerosis (MS), coeliac disease (CD) and Wegener's granulomatosis (WG).

The present invention also provides the use of a composition sufficient to reduce or deplete antibody producing cells and/or prevent expansion of said cells in the manufacture of a medicament for the treatment and/or prevention of T cell mediated autoimmune disease. For example, the medicament can be administered immediately prior to or concomitant with an autoimmune response such as indicated by expansion of a population of T cells and/or B cells and/or by the production of autoantibodies (e.g., expansion of cytotoxic T cells against pancreatic β-islet cells and/or autoantibodies against one or more pancreatic β-islet cell markers in the onset or progression of type 1 diabetes) and/or by an increase in serum glucose levels.

The present invention also provides a composition sufficient to reduce or deplete antibody producing cells and/or prevent expansion of said cells for use in the treatment and/or prevention of T cell mediated autoimmune disease. Mutatis mutandis, the present invention also provides said composition when used to treat and/or prevent T cell mediated autoimmune disease, and/or when administered to a subject suffering from or at risk of suffering from T cell mediated autoimmune disease. For example, the composition can be administered immediately prior to or concomitant with an autoimmune response such as indicated by expansion of a population of T cells and/or B cells and/or by the production of autoantibodies (e.g., expansion of cytotoxic T cells against pancreatic β-islet cells and/or autoantibodies against one or more pancreatic β-islet cell markers in the onset or progression of type 1 diabetes) and/or by an increase in serum glucose levels.

In each of the foregoing embodiments, it is preferred that the composition reduces or depletes antibody producing cells (B cells) in a B cell producing tissue or organ and/or B cell producing tissue or organ of the subject e.g., a composition comprising a compound selected from the group consisting of BCMA-Ig, TACI and BR3-Ig and mixtures thereof. Other compounds such as those identified by a screening method or process described herein to reduce or deplete antibody producing cells (B cells) are not excluded. Alternatively or in addition, it is preferred that the T cell mediated autoimmune disease in each of the foregoing embodiments is diabetes, e.g., type 1 diabetes.

In preferred embodiments of the present invention as described supra, there is also provided a method for preventing type 1 diabetes or reducing type 1 diabetes disease progression in a subject in need thereof, said method comprising administering to a subject an amount of a compound that reduces or depletes antibody producing cells to thereby reduce the number of antibody producing cells and/or prevent expansion of said cells thereby preventing type 1 diabetes or reducing type 1 diabetes disease progression.

As used herein, the term “type 1 diabetes” or “insulin dependent diabetes” or “insulin dependent diabetes mellitus” or “IDDM” shall be taken to mean a diabetes that is characterized by an immune response against an antigen produced by or presented on a pancreatic β islet cell. Preferably, the immune response is sufficient to kill a significant proportion of pancreatic β-islet cells in a type 1 diabetic subject (e.g., at least about 60% or 70% or 80% or 90% of pancreatic β-islet cells are killed relative to the number in a subject that does not suffer from diabetes). As a consequence of the death of pancreatic β-islet cells, type 1 diabetes is characterized by reduced levels of naturally occurring insulin (i.e., endogenous insulin) relative to the level of endogenous insulin in a normal and/or healthy individual.

In a preferred embodiment, a subject suffering from type 1 diabetes has one or more of the following characteristics:
Fasting plasma glucose of greater than or equal to 126 mg/dl with symptoms of diabetes.
Casual plasma glucose (taken at any time of the day) of greater than or equal to 200 mg/dl with the symptoms of diabetes.
Oral glucose tolerance test (OGTT) value of greater than or equal to 200 mg/dl measured at a two-hour interval. The OGTT is given over a three-hour time span.

As used herein, the term “symptoms of diabetes” shall be taken to mean one or more of the following symptoms:
increased blood sugar levels;
increased urine sugar levels;
a unusual thirst
frequent urination
extreme hunger but loss of weight
blurred vision
nausea and/or vomiting
extreme weakness and tiredness
irritability and mood changes
abnormal pancreatic β cell function, e.g., as determined using a standard assay, such as, for example, Homeostasis Model Assessment (HOMA).

Preferably, the immune response against an antigen produced by or presented on a pancreatic β islet cell comprises a B-cell response. In this regard, it is preferable that the immune response is characterized by an increase in the proliferation of B-cells in a subject. Such a proliferation may be accompanied by an increase in production of antibodies that bind to a marker of type 1 diabetes, such as, for example, insulin or a fragment or epitope thereof, proinsulin or a fragment or epitope thereof, IA-2 or a fragment or epitope thereof or glutamic acid decarboxylase (GAD65) or a fragment or epitope thereof.

A treatment that “prevents type 1 diabetes” inhibits the onset of one or more detectable symptoms of diabetes, such as, for example, a symptom described herein. Preferably, such a treatment prevents or reduces the number or proportion of pancreatic β-islet cells killed by an immune response in a subject against an antigen produced by or presented on a pancreatic β islet cell in a that subject. In this regard, it is generally considered that approximately 80% to 90% of β-islet cells are killed in the pancreas of a diabetic subject at the time of disease detection. Methods for determining the level of a detectable symptom of diabetes and/or the number of pancreatic β-islet cells in a subject will be apparent to the skilled person and/or described herein.

By “reducing type 1 diabetes disease progression” is meant that a treatment reduces the severity of type 1 diabetes in a subject. Such a reduction in severity may be, for example, prevention of one or more complications of diabetes, such as, for example, hypoglycemia, hyperglycemia, diabetic ketoacidosis, retinopathy, cataracts, hypertension, renal failure, coronary artery disease, peripheral vascular disease, neuropathy (e.g., peripheral neuropathy or autonomic neuropathy) or increased risk of infection. Alternatively, or in addition, a reduction in severity of type 1 diabetes is characterized by a reduction in the requirement for therapeutic treatment (e.g., insulin administration) or the regularity of therapeutic treatment of a subject compared to a subject that has not received treatment using the method of the invention. Alternatively, or in addition, “reducing type 1 diabetes disease progression” is a delay in the onset of one or more detectable symptoms of diabetes compared to a diabetic subject that has not received treatment with a compound the reduces type 1 diabetes disease progression.

As used herein, the term “a subject in need thereof” is meant a subject that is likely to develop one or more symptoms of diabetes (e.g., as described herein) or is likely to develop type 1 diabetes or is at risk of developing one or more symptoms of diabetes or is at risk of developing type 1 diabetes. In this respect, a subject at risk of developing type 1 diabetes or likely to develop type 1 diabetes is likely to develop autoimmunity against β islet cells including a transplanted β islet cell. For example, such a subject has a family history of type 1 diabetes, or is from a population with increased risk of type 1 diabetes, or has developed or is developing an immune response that is characteristic of type 1 diabetes (e.g., auto-antibodies against insulin or pro-insulin or IA-2 or GAD65). Suitable methods for determining a subject that is likely to develop type 1 diabetes or is at risk of developing type 1 diabetes will be apparent to the skilled person and/or described herein. Accordingly, in one embodiment, the method of the invention comprises determining a subject in need of treatment.

Alternatively, or in addition, a subject in need thereof presenting with hyperglycemia and/or polyuria and/or polydipsia and/or any other manifestation of diabetes. Other a subject in need of treatment presents with abnormal β-cell functions, for example, as determined by an assay known in the art, such as, for example, a homeostasis model assessment (HOMA).

Preferably a subject in need of treatment has an increased level of blood glucose compares to, for example, a normal or healthy subject and/or a blood glucose level detected in the subject previously. For example, the subject has a fasting blood glucose level between about 100 mg/dL to about 125 mg/dL. Alternatively, or in addition, the subject has a glucose tolerance of about 140 mg/dL to about 199 mg/dL.

As will be apparent to the skilled artisan from the foregoing a “subject in need thereof” includes a subject that has received a β-islet cell transplant, e.g., for the treatment of type 1 diabetes, and subsequently develops an autoimmune response against the transplanted cells. Such an autoimmune response is detected, for example, by performing a method described herein according to any embodiment. For example, the autoimmune response is detected by detecting an autoantibody that binds to a pancreatic β-islet cell and/or an antigen thereof in a sample. Alternatively, or in addition, an autoimmune response is detected by detecting a T cell capable of binding to a pancreatic β islet cell or an antigen thereof (e.g., an islet-specific glucose-6-phosphatase-related protein (IGRP) or a fragment or epitope thereof). Alternatively, or in addition, an autoimmune response is detected by detecting a B cell expansion in a sample from a subject.

As used herein, the term “a compound that reduces or depletes antibody producing cells” shall be taken to mean a compound that binds to and kills a B cell or a B cell precursor and/or a compound that inhibits the expression and/or activity of a peptide, polypeptide or protein or other cellular component that is required for B cell development, B cell division and/or B cell survival.

Administration of a compound that reduces or depletes antibody producing cells demonstrably reduces the number of antibody producing cells in a subject. For example, administration of such a compound reduces the number of mature B cells in the blood of a subject and/or in the spleen of a subject.

For example, administration of a compound that reduces or depletes antibody producing cells results in a reduction in antibody producing cells in a subject by about 40% or more. Alternatively, administration of a compound that reduces the number of antibody producing cells results in a reduction in antibody producing cells in a subject by about 50% or more. Alternatively, administration of a compound that reduces the number of antibody producing cells results in a reduction in antibody producing cells in a subject by about 60% or more. Alternatively, administration of a compound that reduces the number of antibody producing cells results in a reduction in antibody producing cells in a subject by about 70% or more. Alternatively, administration of a compound that reduces the number of antibody producing cells results in a reduction in antibody producing cells in a subject by about 80% or more. Alternatively, administration of a compound that reduces the number of antibody producing cells results in a reduction in antibody producing cells in a subject by about 90% or more.

In this regard, the “compound” may be a single compound or alternatively, may be a plurality of compounds administered individually or in a single composition, e.g., a pharmaceutical composition.

Suitable compounds will be apparent to the skilled person and/or described herein. For example, a suitable compound comprises an antibody or an antigen binding region thereof capable of binding to, for example, a B-cell marker such as, for example, CD-19, CD-20, CD-22, CD-37. Alternatively, a suitable compound comprises, for example, a protein or fragment such as, for example, B-cell maturation antigen (BCMA) or transmembrane activator and calcium modulator and cyclophilin ligand (CAML) interactor (TACI) or BAFF-receptor fused to a fragment of an antibody or immunoglobulin, such as, for example, a Fc region of an immunoglobulin. Suitable compounds will be apparent to the skilled person and/or described herein.

For example, a suitable compound binds to a protein expressed on the surface of a B cell and prevents B cell development and/or kills the B cell.

Alternatively, the compound binds to a protein required for B cell development and/or B cell survival to thereby reduce the number of antibody producing cells in the subject. For example, the compound binds to a B-cell-activating-factor-belonging-to-the-TNF-family (BAFF) polypeptide to thereby reduce the number of antibody producing cells in the subject. For example, the compound is a fusion protein comprising an extracellular domain of a BAFF-receptor and a Fc domain of human immunoglobulin G. As exemplified herein, a suitable compound the compound is a fusion protein comprising an extracellular domain of a B-cell maturation antigen (BCMA) polypeptide and a Fc domain of human immunoglobulin G.

For example, the method described herein according to any embodiment comprises administering the compound to the subject immediately prior to or concomitantly with the onset of an immune response by the subject against a pancreatic β-islet cell.

As used herein, the term “immediately prior to” shall be taken to mean that the number of antibody producing cells are reduced at a time before but sufficiently close to the time of the immune response (preferably, B-cell expansion) to ensure that an antibody response against an antigen associated with type 1 diabetes, or B-cell proliferation does not occur or is reduced.

As used herein, the term “concomitantly with” shall be taken to mean that the compound that depletes or reduces antibody producing cells is administered at the time of the immune response against a pancreatic β-cell. Preferably, the compound is administered at the time of B-cell expansion that accompanies or is characteristic of an immune response against a pancreatic β-cell. In this regard, the compound need not be administered at exactly the time of B-cell expansion. Rather, the compound need only be administered at about this time (e.g., at the time of detection of a significantly increased number of B-cells and/or a significantly increased level of an antibody against an antigen associated with type 1 diabetes relative to a suitable reference sample).

In the case of diabetes or treatment of a complication of diabetes (e.g., by pancreatic β islet cell transplant or pancreatic graft) the compound that depletes or reduces antibody producing cells is preferably administered at the time of an increase in serum glucose levels (e.g., hyperglycemia), e.g., a spike in serum glucose levels. In this respect, the present inventors have discovered that such an increase in blood glucose levels corresponds to a period in which B cell and/or T cell expansion occurs in a subject. Such an assay is relatively easy and inexpensive to perform to determine a suitable time to administer a compound to depletes or reduces antibody producing cells to a subject.

Preferably, the compound is administered to a subject having a fasting blood glucose level of at least about 100 mg/dL to about 125 mg/dL.

Preferably, the compound is administered to a subject suffering from polydipsia and/or polyuria and/or abnormal pancreatic β cell function, e.g., as assessed by a test known in the art, such as for example, HOMA.

To permit suitable timing of administration of a compound, a method according to the any embodiment of the invention additionally comprises detecting the onset of the immune response against a pancreatic β-islet cell or predicting the onset of the immune response against a pancreatic β-islet cell prior to administration of the compound.

For example, a method for detecting the onset of the immune response against a pancreatic β-islet cell comprises:
(i) contacting an immunoglobulin containing sample from the subject with a sample comprising pancreatic β cell and/or with a protein expressed by a pancreatic β-cell or an immunogenic fragment or epitope thereof for a time and under conditions sufficient for an antigen-antibody complex to form; and
(ii) detecting the antigen-antibody complex,
   wherein detection of the antigen-antibody complex is indicative of the onset of an immune response against a pancreatic β-islet cell by the subject.

Alternatively, a method for detecting the onset of the immune response against a pancreatic β-islet cell comprises:
(i) contacting a T cell containing fraction from the subject with a protein expressed by a pancreatic β-cell or an immunogenic fragment or epitope thereof or a protein complex comprising said protein, fragment and/or epitope for a time and under conditions sufficient for a T cell to bind to the protein, fragment, epitope or complex; and
(ii) detecting the T cell bound to the protein, fragment, epitope or complex, wherein detection of the T cell bound to the protein, fragment, epitope or complex is indicative of the onset of an immune response against a pancreatic β-islet cell by the subject.

For example, such a method comprises contacting the T cell fraction with a protein complex comprising an islet-specific glucose-6-phosphatase-related protein or an immunogenic fragment or epitope thereof. Preferably, the method comprises detecting a complex of said glucose-6-phosphatase-related protein and/or immunogenic fragment and/or epitope thereof. For example, the method comprises contacting the T cell fraction with a multimer, e.g., a tetramer of a glucose-6-phosphatase-related protein and/or immunogenic fragment and/or epitope thereof.

Alternatively, a method for detecting the onset of the immune response against a pancreatic β-islet cell comprises:
(i) determining the number of B cells in a sample from a subject suspected of suffering from or at risk of suffering from type 1 diabetes; and
(ii) comparing the number of B cells determined at (i) to the number of B cells in a reference sample,
   wherein an increased number of the B cells or the type of B cell at (i) compared to (ii) is indicative of the onset of an immune response against a pancreatic β-islet cell by the subject.

For example, such a method comprises determining the number of marginal-zone (MZ) B cells in the sample from the subject and comparing the number of MZ B cells in the sample from the subject to the number of MZB cells in a reference sample.

For example, the B cells and/or B cell counts are determined in a whole blood sample or an extract or a fraction thereof. Alternatively, B cells and/or B cell counts are determined in a spleen or a fragment thereof or an extract or a fraction thereof.

A suitable reference sample for determining onset of an immune response against a pancreatic β islet cells is selected from the group consisting of:
(i) a sample from a normal subject;
(ii) a sample from a healthy subject;
(iii) a sample or data set comprising measurements for the subject being tested wherein said sample or measurements have been taken previously, such as, for example, when the subject was known to healthy or, in the case of a subject having the disease, when the subject was diagnosed or at an earlier stage in disease progression;
(iv) an extract of any one of (i) to (iii);
(v) a fraction of any one of (i) to (iii);
(vi) a data set comprising measurements of the number of B cells in a sample from a healthy individual or a population of normal individuals;
(vii) a data set comprising measurements of the number of B cells in a sample from a normal individual or a population of normal individuals; and
   For example, the reference sample is (i) or (ii)m described above.

Following methods known in the art, a skilled person could readily perform and/or obtain reagents to perform a method described herein according to any embodiment to determine a suitable time to administer a compound that depletes or reduced antibody producing cells to a subject.

The method described herein according to any embodiment may additionally comprising ceasing administering the compound to the subject following administration of the compound for a time sufficient to reduce the number of antibody producing cells in the subject. Such a step permits the subject to re-develop antibody-producing cells, and, as a consequence, the subject is not immune suppressed.

The present invention additionally provides a method described herein according to any embodiment additionally comprising determining the number of antibody producing cells in the subject following administration of the compound and ceasing administering the compound if the number of antibody producing cells is sufficiently reduced to prevent type 1 diabetes or reduce type 1 diabetes disease progression.

The present invention also provides a method for preventing type 1 diabetes onset in a subject in need thereof, said method comprising administering to the subject an amount of a fusion protein comprising an extracellular domain of a B-cell maturation antigen (BCMA) polypeptide and a Fc domain of human immunoglobulin G (Ig) to thereby reduce the number of antibody producing cells and/or prevent expansion of said cells, wherein said compound is administered immediately prior to or concomitant with the onset of an autoimmune response against a pancreatic β-islet cell as determined by expansion of cytotoxic T cells against pancreatic β-islet cells and/or autoantibodies against one or more pancreatic β-islet cell markers, thereby preventing type 1 diabetes onset.

The present invention also provides for the use of a compound that reduces the number of antibody producing cells and/or prevents expansion of said cells in the manufacture of a medicament for the prevention of type 1 diabetes or for the reduction of type 1 diabetes disease progression.

Furthermore, the present invention provides for the use of a compound that reduces the number of antibody producing cells and/or prevents expansion of said cells in the manufacture of a medicament for the prevention of type 1 diabetes or for the reduction of type 1 diabetes disease progression, said medicament being for administration to a subject immediately prior to or concomitantly with the onset of an immune response against a pancreatic β-islet cell.

For example, the compound is a fusion protein (BCMA-Ig) comprising an extracellular domain of a B-cell maturation antigen (BCMA) polypeptide and a Fc domain of human immunoglobulin G (Ig).

The present invention also provides a compound that reduces the number of antibody producing cells and/or prevents expansion of said cells for use in the prevention of type 1 diabetes or in the reduction of type 1 diabetes disease progression.

The present invention also provides a compound that reduces the number of antibody producing cells and/or prevents expansion of said cells when administered to a subject immediately prior to or concomitantly with the onset of an immune response against a pancreatic β-islet cell to prevent type 1 diabetes or to reduce type 1 diabetes disease progression.

For example, the compound is a fusion protein comprising an extracellular domain of a B-cell maturation antigen (BCMA) polypeptide and a Fc domain of human immunoglobulin G.

### BRIEF DESCRIPTION OF THE DRAWINGS

FIG. 1A is a graphical representation showing total numbers of splenocytes in female NOD mice (), age-matched non-diabetic C57BL/6 (◯), BALB/c (Δ) and DBA (□) mice. Results represent values from individual mice (n=6 per group), bar represents median value. *** (p<0.001).

FIG. 1B is a graphical representation showing absolute numbers of B lymphocytes in the spleen of female NOD mice (), age-matched non-diabetic C57BL/6 (◯), BALB/c (Δ) and DBA (□) mice. Results represent values from individual mice (n=6 per group), bar represents median value. ** (p<0.01), *** (p<0.001).

FIG. 1C is a graphical representation showing total numbers of T lymphocytes in the spleen of female NOD mice (), age-matched non-diabetic C57BL/6 (◯), BALB/c (Δ) and DBA (□) mice. Results represent values from individual mice (n=6 per group), bar represents median value. * (p<0.05), *** (p<0.001).

FIG. 1D Follicular B (FoB) cells in the spleen of female NOD mice (), age-matched non-diabetic C57BL/6 (◯), BALB/c (Δ) and DBA (□) mice. Results represent values from individual mice (n=6 per group), bar represents median value. *** (p<0.001).

FIG. 1E is a graphical representation showing absolute numbers of Marginal Zone B (MZB) cells in the spleen of female NOD mice (), age-matched non-diabetic C57BL/6 (◯), BALB/c (Δ) and DBA (□) mice. Results represent values from individual mice (n=6 per group), bar represents median value. *** (p<0.001).

FIG. 2A is a graphical representation showing the percentage of mature and transitional type 1 cells in the blood as a proportion of all lymphocytes for NOD (filled bar) and C57BL/6 (open bar) mice. Values represent mean percentage of B lymphocytes±sem, n=4 per group.

FIG. 2B is a graphical representation showing absolute numbers of B lymphocytes precursors in the bone marrow (tibia plus femur) for NOD () and C57BL/6 (◯) mice. Results represent values from individual mice (n=6 per group), bar represents median value.

FIG. 3A is a copy of a photographic representation showing results of a Western Blot to detect total phosphorylated tyrosine residues to detect the level of phosphorylation of signaling proteins in purified B lymphocytes from NOD (N) and C57BL/6 (B) mice stimulated with 10 μg/ml anti-IgM for 5 minutes. C represents unstimulated cells and IgM represents stimulated cells.

FIG. 3B is a copy of a photographic representation showing results of a Western Blot to detect total phosphorylated tyrosine residues in B lymphocytes from hen egg lysozyme (HEL) specific B Cell Receptor transgenic NOD.IgHEL (N) and C57BL/6.IgHEL (B) mice stimulated with increasing concentrations of HEL peptide for 5 minutes.

FIG. 4A is a graphical representation showing results of Fluorescence Activated Cell Sorting (FACS) analysis of splenic lymphocytes demonstrating expanded MZB cell population in NOD, BAFF-Transgenic (BAFF-Tg), TACI Knockout (TACI) and C57/BL6 mice. The numbers depicted represent percentage of FoB and MZB cells within each gate as a fraction of the total lymphocytes. One representative experiment of six is shown.

FIG. 4B is a graphical representation showing BAFF serum concentrations in NOD mice, diabetic NOD mice, C57Bl/6 mice and Balb/c mice. Data represents mean±standard error of the mean, n=5 per group.

FIG. 4C is a graphical representation showing the level of expression of BAFF-R, BCMA and TACI receptors on NOD (black line) and C57BL/6 (grey line) B lymphocytes. One representative experiment of three is shown.

FIG. 5A is a graphical representation showing the results of semi-quantitative RT-PCR analysis of S1P1 and S1P3 mRNA expression in FACS purified FoB (▪ or □) or MZB ( or ◯) isolated from NOD (filled symbol) or C57BL/6 (open symbol) mice. Results represent fold change in S1P receptor expression relative to GAPDH, for individual mice, n=3 per group, bar represents median value. ** (p<0.01), *** (p<0.001).

FIG. 5B is a graphical representation showing the chemotactic response of FACS purified NOD (circle symbol) and C57BL/6 (triangle symbol) FoB cells to increasing concentrations of S1P with () or without (◯) FTY720 treatment. Results represent mean±sem of one of four experiments performed in triplicate.

FIG. 5C is a graphical representation showing the chemotactic response of FACS purified NOD (circle symbol) and C57BL/6 (triangle symbol) MZB cells to increasing concentrations of S1P with () or without (◯) FTY720 treatment. Results represent mean±sem of one of four experiments performed in triplicate. *** (p<0.001).

FIG. 6A is a graphical representation showing Ova-specific T dependent (TD) antibody responses for NOD (◯) or C57BL/6 () mice. Results represent individual values from 10 mice per group at day 28 post-immunization, bar represents median value. * (p<0.05), ** (p<0.01), *** (p<0.001).

FIG. 6B is a graphical representation showing Ficoll-specific T independent (TI) antibody responses for NOD (◯) or C57BL/6 () mice. Results represent individual values from 10 mice per group at day 14 post-immunization, bar represents median value. ** (p<0.01), *** (p<0.001).

FIG. 7A is a graphical representation showing CD40 expression on splenic B lymphocytes from NOD (black line) and C57BL/6 (grey line) mice. A representative experiment of six is shown.

FIG. 7B is a graphical representation showing the level of proliferation of splenic B lymphocytes from NOD (filled bars) or C57BL/6 (open bars) mice in response to anti-CD40 (1 μg/ml), IL-4 (100 ng/ml), anti-CD40 (1 μg/ml) plus IL-4 (100 ng/ml), anti-μ (20 μg/ml), bacterial DNA (CpG) (3 μg/ml) or LPS (500 ng/ml) (as indicated on the X-axis). Results represent mean stimulation index (SI)±standard error of the mean of one of three experiment conducted in triplicate. * (p<0.05), ** (p<0.01), *** (p<0.001).

FIG. 8A is a graphical representation showing absolute numbers of B (circle symbol) and T (square symbol) splenocytes in NOD mice at various ages ranging from 5 weeks of age to 25 weeks of age. Results represent mean±standard error of the mean values from at least n=6 mice per time point. ** (p<0.01), *** (p<0.001).

FIG. 8B is a graphical representation showing absolute numbers of B (circle symbol) and T (triangle symbol) lymphocytes in the pancreatic lymph nodes of NOD mice at various ages ranging from 5 weeks of age to 25 weeks of age. Results represent mean±standard error of the mean values from at least n=6 mice per time point. ** (p<0.01), *** (p<0.001).

FIG. 8C is a graphical representation showing absolute numbers of CD4+ (circle symbol) and CD8+ (triangle symbol) T cells in spleens of NOD mice at various ages ranging from 5 weeks of age to 25 weeks of age. Results represent mean±standard error of the mean values from at least n=6 mice per time point. ** (p<0.01), *** (p<0.001).

FIG. 8D is a graphical representation showing absolute numbers of CD4+ (circle symbol) and CD8+ (triangle symbol) T cells in pancreatic lymph nodes of NOD mice at various ages ranging from 5 weeks of age to 25 weeks of age. Results represent mean±standard error of the mean values from at least n=6 mice per time point. ** (p<0.01), *** (p<0.001).

FIG. 8E is a graphical representation showing numbers of FoB cells, MZB cells, T2 cells and T1 cells as indicated, in the spleen of NOD mice at various ages ranging from 5 weeks of age to 25 weeks of age. Results represent mean±standard error of the mean values from at least n=6 mice per time point.

FIG. 8F is a graphical representation showing numbers of FoB cells, MZB cells, T2 cells and T1 cells as indicated, in the pancreatic lymph nodes of NOD mice at various ages ranging from 5 weeks of age to 25 weeks of age. Results represent mean±standard error of the mean values from at least n=6 mice per time point.

FIG. 9A is a graphical representation showing FACS analysis of B lymphocyte subsets found infiltrating the pancreas of NOD mice. One representative experiment of at least six is shown.

FIG. 9B is a graphical representation showing FACS analysis of B lymphocyte subsets in the spleen of NOD mice. One representative experiment of at least six is shown.

FIG. 10A is a graphical representation showing the number of B cells in the blood and spleen (as indicated at the left-hand side of the figure) of NOD mice during and after treatment with BCMA-Fc, Control levels are also indicated. Boxed regions indicated B cells. The number depicted represents the percentage of total cells detected that are B cells. One experiment of six is shown.

FIG. 10B is a graphical representation showing a Kaplan-Meier cumulative survival plots for NOD mice administered BCMA-Fc (black line, n=10), IVIg (grey line, n=20) or PBS (broken line, n=30) from 9-15 weeks-of-age. **p=0.0021 for BCMA-Fc treatment versus IVIg (Mantel-Cox Log-Rank analysis)

FIG. 10C is a graphical representation showing a Kaplan-Meier cumulative survival plots for NOD mice administered BCMA-Fc (black line, n=10), IVIg (grey line, n=20) from 4-6 weeks-of-age.

FIG. 10D is a graphical representation showing a Kaplan-Meier cumulative survival plots for NOD mice administered BCMA-Fc (black line, n=12), IVIg (grey line, n=20) from 12-18 weeks-of-age.

FIG. 11A is a graphical representation showing the incidence of diabetes in NOD.SCID mice following adoptive transfer of splenocytes from diabetes free BCMA-Fc treated NOD mice (square symbol, n=9), newly-diabetic NOD mice (Δ, n=23), or a 1:1 ratio of splenocytes from BCMA-Fc treated and newly-diabetic NOD mice (◯, n=10). Differences in diabetes incidence for NOD.SCID mice receiving splenocytes from protected mice are significant by Mantel-Cox Log-Rank analysis (p=0.0140).

FIG. 11B is a graphical representation showing that BCMA-Fc treated and protected mice are not immune suppressed. Ova specific TD response of NOD mice treated with BCMA-Fc (150 μg twice weekly) for 9-15 weeks (◯, n=4) at 50 weeks-of-age versus control IVIg treated NOD mice (, n=4). Results represent individual values indicating total TD immunoglobulin production from 4 mice per group at day 28, bar represents median value. *** (p<0.001).

FIG. 12 is a graphical representation showing the frequency of IGRP+ T cells increases in NOD mice at 7, 10 and 16 weeks of age. Each point indicates percentage IGRP+ CD8 T cells from one individual mouse. Bars indicate mean value. IGRP+ cells were detected by IGRP-tetramer staining gated on CD8+ splenocytes.

### DETAILED DESCRIPTION OF THE PREFERRED EMBODIMENTS

### 1. Suitable Subjects

In a preferred embodiment, the method of the invention is a method of prophylaxis of T cell-mediated autoimmune disease, such as, type 1 diabetes or rejection of a pancreatic islet cell graft or rejection of a whole pancreas transplant, i.e., the method is used to prevent the onset of T cell-mediated autoimmune disease, such as, type 1 diabetes or rejection of a pancreatic islet cell graft or rejection of a whole pancreas transplant or substantially reduce the symptoms associated with T cell-mediated autoimmune disease, such as, type 1 diabetes or rejection of a pancreatic islet cell graft or rejection of a whole pancreas transplant.

Furthermore, it is preferable, that the subject has not undergone sufficient physiological changes to develop T cell-mediated autoimmune disease, such as, type 1 diabetes or rejection of a pancreatic islet cell graft or rejection of a whole pancreas transplant. For example, the subject comprises or has retained sufficient pancreatic β-islet cells to produce sufficient insulin to avoid onset of or to reduce or avoid the symptoms associated with type 1 diabetes. Accordingly, it is preferable that the subject has not raised an immune response against a pancreatic β-cell.

In a preferred embodiment, the subject is at risk of developing T cell-mediated autoimmune disease, such as, type 1 diabetes or rejection of a pancreatic islet cell graft or rejection of a whole pancreas transplant. For example, the subject has a family history of T cell-mediated autoimmune disease, such as, type 1 diabetes or rejection of a pancreatic islet cell graft or rejection of a whole pancreas transplant. For example, a subject that has a parent or sibling that suffers from type 1 diabetes has approximately a 2% to 6% chance of developing type 1 diabetes. However, should both parents be diabetic (i.e., suffer from type 1 diabetes), a subject has about a 30% chance of developing type 1 diabetes.

In another embodiment, a subject carrying an allele that confers susceptibility or is indicative of susceptibility to a T cell-mediated autoimmune disease, such as, type 1 diabetes or rejection of a pancreatic islet cell graft or rejection of a whole pancreas transplant is a subject suitable for treatment. For example, a subject that expresses either or both HLA Class II molecule DR3 or DR4 has an increased risk of developing type 1 diabetes.

Subjects carrying a mutation in the Sumo-4 gene that increases activity of the encoded protein have an increased risk of developing type 1 diabetes (Guo et al., Nat. Genet. 36: 837-841, 2004). Similarly, a subject carrying a polymorphism in one or more of the following genes has an increased risk of developing type 1 diabetes:

(i) a TAB2 gene and/or a NF kappaB gene (Kosoy and Cancannon, Genes and Immunology, 6: 231-235, 2005);
(ii) a CBLB gene (Kosoy et al., Genes and Immunology, 5: 232-235, 2004);
(iii) a PTPN22 gene (Onengut-Gumuscu et al., Genes and Immunology, 5: 678-680, 2004); or
(iv) a protein kinase C β1 gene (Araki et al., J Am Soc Nephrol. 14:2015-24, 2003);

Alternatively, or in addition, a subject that suffered from or suffers from (i.e., has a history of) an increased incidence of viral infections is at risk of developing a T cell-mediated autoimmune disease, such as, type 1 diabetes or rejection of a pancreatic islet cell graft or rejection of a whole pancreas transplant. For example, viruses that have been associated with type 1 diabetes include, for example, coxsackie B virus, an enterovirus, an adenovirus, a rubella virus, a cytomegalovirus, and an Epstein-Barr virus.

Dietary factors have also been associated with an increased risk of developing type 1 diabetes. For example, research by Kimpimaki et al., Diabetologia. 44: 63-9, 2001 indicates that breastfeeding at least three months decreases the risk of type 1 diabetes. Some studies have also found that exposure to cow's milk or cow's milk-based formula before one year of age may increase diabetes risk (Vaarala et al., Diabetes. 48: 1389-94, 1999).

Additional risk factors for type 1 diabetes include, for example, the subject suffering from another autoimmune disease, exposure to streptozotocin or RH-787, or the subject suffering from a Chromosomal abnormality, such as, for example, Down syndrome, Turner syndrome, Klinefelter syndrome or Prader-Willi syndrome.

### 2. Suitable Compounds

Any compound that is capable of reducing the number of antibody producing cells in a subject is suitable for use in the method of the present invention. Preferably, the compound reduces the number of B cells in a subject. More preferably, administration of the compound results in demonstrable B cell depletion, even more preferably, administration of the compound will result in a depletion of B cell number by about 50%, or 60% or 70% or more after several days.

Suitable compounds will be apparent to the skilled person and include, for example, an antibody, an antibody fragment, an antibody conjugate, a peptide or protein, a peptide or protein conjugate, a nucleic acid molecule or a small molecule.

Such a compound may be conjugated to a cytotoxic compound, e.g., as described herein, to thereby facilitate depletion of antibody producing cells from a subject. Alternatively, or in addition, the compound may inhibit the activity of a molecule, e.g., a protein that is required for B cell development. Alternatively, or in addition the compound may induce cell death by, for example, antibody-dependent cell-mediated toxicity or complement dependent cell death.

“Antibody-dependent cell-mediated cytotoxicity” and “ADCC” refer to a cell mediated reaction in which nonspecific cytotoxic cells that express a Fc receptor (FcR) (e.g., a Natural Killer (NK) cell, a neutrophil or a macrophage) recognize bound antibody on a target cell and subsequently cause lysis of the target cell. The primary cells for mediating ADCC, NK cells, express FcyRIII only, whereas monocytes express FcyRI, FcyRII and FcyRIII. To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Natl. Acad. Sci. USA, 95:652-656, 1998.

“Complement dependent cytotoxicity” or “CDC” refers to the ability of a molecule to lyse a target in the presence of complement. The complement activation pathway is initiated by the binding of the first component of the complement system (Clq) to a molecule (e.g., an antibody) complexed with a cognate antigen. To assess complement activation, a CDC assay, e.g. as described in Gazzano-Santoro et al., J. Immunol. Methods 202:163, 1996, may be performed.

### 2.1 Antibodies

As used herein the term “antibody” refers to intact monoclonal or polyclonal antibodies, immunoglobulin (IgA, IgD, IgG, IgM, IgE) fractions, humanized antibodies, or recombinant single chain antibodies, as well as fragments thereof, such as, for example Fab, F(ab)2, and Fv fragments.

Antibodies referred to herein are obtained from a commercial source, or alternatively, produced by conventional means.

For example, rituximab (RITUXAN™) antibody is a genetically engineered chimeric murine/human monoclonal antibody directed against human CD20 antigen (commercially available from Genentech, Inc., South San Francisco, Calif., U.S.). Rituximab is the antibody referred to as “C2B8” in U.S. Pat. No. 5,736,137. This antibody has been shown to be capable of binding to and inducing cell death in B-cells. In vitro mechanism of action studies have demonstrated that rituximab binds human complement and lyses lymphoid B cell lines through complement-dependent cytotoxicity (CDC) (Reff et al. Blood 83: 435-445, 1994). Additionally, this antibody has significant activity in assays for antibody-dependent cellular cytotoxicity (ADCC). In vivo preclinical studies have shown that rituximab depletes B cells from the peripheral blood, lymph nodes, and bone marrow of cynomolgus monkeys, presumably through complement and cell-mediated processes (Reff et al., supra).

Additional anti-CD20 antibodies include, for example, the murine antibody Zevalin™ which is linked to the radioisotope, Yttrium-90 (IDEC Pharmaceuticals, San Diego, Calif.), Bexxar™, which is a another fully murine antibody conjugated to I-131 (Corixa, Wash.).

Anti-CD22 antibodies have also been shown to be useful for depleting B-cells in a subject. CD22 is a B-cell-specific molecule involved in B-cell adhesion that may function in homotypic or heterotypic interactions (Stamenkovic et al, Nature 344: 74 1990). For example, U.S. Pat. No. 5,484,892, describes monoclonal antibodies that bind CD22 with high affinity and block the interaction of CD22 with other ligands.

U.S. Pat. No. 5,789,557, discloses chimeric and humanized anti-CD22 monoclonal antibodies produced by CDR grafting and the use thereof in conjugated and unconjugated form for therapy and diagnosis of B-cell lymphomas and leukemias. The reference discloses especially such antibodies conjugated to cytotoxic agents, such as chemotherapeutic drugs, toxins, heavy metals and radionuclides.

Furthermore, PCT applications WO 98/42378, WO 00/20864, and WO 98/41641 describe monoclonal antibodies, conjugates and fragments specific to CD22 and therapeutic use thereof.

An anti-human CD22 monoclonal antibody of the IgG1 isotype is also commercially available from Leinco Technologies.

Anti-CD 19 antibodies are also useful for the depletion of B-cells in a subject. For example, U.S. Pat. No. 5,686,072 discloses the use of anti-CD19 and anti-CD22 antibodies and immunotoxins for B-cell depletion.

Anti-CD23 antibodies have also been shown to be useful for depleting B-cells in a subject. Specific examples of antibodies that bind CD23 are known in the art. For example, a Primatized™ antibody specific to human CD23 is described in U.S. Pat. No. 6,011,138; an antibody specific to human CD23 is described in Rector et al. J. Immunol. 55:481-488, 1985; or Flores-Rumeo et al. Science 241:1038-1046, 1993.

Alternatively, or in addition, the antibody or fragment thereof is produced using a method known in the art. Typically, such an antibody will be capable of specifically or selectively binding to a marker or antigen that is specific to or increased at an increased level by an antibody producing cell, e.g., a B cell. Exemplary B cell markers include, for example, CD10, CD19, CD20, CD21, CD22, CD23, CD24, CD37, CD53, CD72, CD73, CD74, CDw75, CDw76, CD77, CDw78, CD79a, CD79b, CD80, CD81, CD82, CD83, CDw84, CD85 or CD86 leukocyte surface marker.

Alternatively, or in addition, the antibody or antibody fragment or antibody conjugate is capable of binding to a molecule that is necessary for production of a B cell, such as, for example, BCMA or TACI or BAFF.

High titer antibodies are preferred, as these are more useful in therapeutic applications. By “high titer” is meant a titer of at least about 1:10³ or 1:10⁴ or 1:10⁵. Methods for determining the titer of an antibody will be apparent to the skilled artisan. For example, the titer of an IgG antibody in purified antiserum may be determined using an ELISA assay to determine the amount of IgG in a sample. Typically an anti-IgG antibody or Protein G is used in such an assay. The amount detected in a sample is compared to a control sample of a known amount of purified and/or recombinant IgG. Alternatively, a kit for determining antibody may be used, e.g. the Easy TITER kit from Pierce (Rockford, Ill., USA).

Antibodies may be prepared by any of a variety of techniques known to those of ordinary skill in the art, and/or described, for example in, Harlow and Lane (In: Antibodies: A Laboratory Manual, Cold Spring Harbor Laboratory, 1988). In one such technique, an immunogen comprising the antigenic polypeptide is initially injected into any one of a wide variety of animals (e.g., mice, rats, rabbits, sheep, humans, dogs, pigs, chickens and goats). The immunogen is derived from a natural source, produced by recombinant expression means, or artificially generated, such as by chemical synthesis (e.g., BOC chemistry or FMOC chemistry).

A peptide, polypeptide or protein is optionally joined to a carrier protein, such as, for example, bovine serum albumin or keyhole limpet hemocyanin. The immunogen and, optionally, a carrier for the protein is injected into the animal host, preferably according to a predetermined schedule incorporating one or more booster immunizations, and blood collected from said the animals periodically. Optionally, the immunogen is injected in the presence of an adjuvant, such as, for example Freund's complete or Freund's incomplete adjuvant, lysolecithin and/or dinitrophenol to enhance the host's immune response to the immunogen. Monoclonal or polyclonal antibodies specific for the polypeptide are then purified from the blood isolated from the host by, for example, affinity chromatography using the polypeptide immunogen coupled to a suitable solid support.

Monoclonal antibodies specific for the antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines are produced, for example, from spleen cells obtained from an animal immunized as described supra. The spleen cells are then immortalized by, for example, fusion with a myeloma cell fusion partner, preferably one that is syngenic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Monoclonal antibodies are isolated from the supernatants of growing hybridoma colonies using methods such as, for example, affinity purification as described supra. In addition, various techniques may be employed to enhance the yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction.

It is preferable that an immunogen used in the production of an antibody is one which is sufficiently antigenic to stimulate the production of antibodies that will bind to the immunogen and is preferably, a high titer antibody. For example, an immunogen may be an entire protein.

Alternatively, an immunogen consists of a peptide representing a fragment of a polypeptide. Preferably, an antibody raised to such an immunogen also recognizes the full-length protein from which the immunogen was derived, such as, for example, in its native state or having native conformation.

As discussed supra antibody fragments are contemplated by the present invention. The term “antibody fragment” refers to a portion of a full-length antibody, generally the antigen binding or variable region. Examples of antibody fragments include Fab, Fab′, F(ab′)₂ and Fv fragments.

Papain digestion of an antibody produces two identical antigen binding fragments, called the Fab fragment, each with a single antigen binding site, and a residual “Fc” fragment.

Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

An “Fv” fragment is the minimum antibody fragment that contains a complete antigen recognition and binding site. This region consists of a dimer of one heavy and one light chain variable domain in a non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen.

A Fab fragment [also designated as F(ab)] also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

“Single-chain Fv” or “scFv” antibody fragments comprise the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv, see Pluckthun in The Pharmacology of Monoclonal Antibodies, vol. 113, Rosenburg and Moore eds. Springer-Verlag, New York, pp. 269-315 (1994).

In a preferred embodiment, the antibody is a chimeric or a humanized antibody. A “chimeric” antibody is an antibody or fragment thereof in which a portion of the heavy and/or light chain is identical with or homologous to corresponding sequences in an antibody derived from a particular species or belonging to a particular antibody class or subclass, while the remainder of the chain(s) is identical with or homologous to corresponding sequence in an antibody derived from another species or belonging to another antibody class or subclass, as well as fragments of such antibodies, so long as they exhibit the desired biological activity (suitable methods for the production of a chimeric antibody are described, for example, in U.S. Pat. No. 4,816,567; or Morrison et al., Proc. Natl. Acad. Sci, USA, 81: 6851-6855, 1984).

A “humanized” antibody is a humanized form forms of a non-human (e.g., murine) antibody. Such an antibody is a chimeric immunoglobulin, immunoglobulin chain or fragments thereof (such as Fv, Fab, Fab′, F(ab′)2 or other antigen-binding subsequence of an antibody) which contains minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and/or capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optimally also will comprise at least a portion of an immunoglobulin constant region (Fc), typically that of a human immunoglobulin. Suitable methods for the production of humanized antibodies are known in the art and/or described, for example, in Jones et al., Nature, 321:522-525, 1986; or Reichmann et al., Nature, 332:323-329, 1988

Human antibodies are also produced using various techniques known in the art, including using a phage-display library (Hoogenboom and Winter, J. Mol. Biol., 227:381, 1991.

In one embodiment, the antibody or fragment thereof is conjugated to a compound, e.g., a cytotoxic compound to thereby enable B-cell depletion in a subject. For example an antibody is conjugated to one or more small molecule toxins, such as, a calicheamicin, a maytansine (U.S. Pat. No. 5,208,020), a trichothene, or s CC 1065.

In one embodiment, the antibody is conjugated to one or more maytansine molecules (e.g. about 1 to about 10 maytansine molecules per antagonist molecule). Maytansine may, for example, be converted to May SS-Me which may be reduced to May-SH3 and reacted with the modified antibody (Charm et al. Cancer Research 52:127-131, 1992) to generate a maytansinoid-antibody conjugate.

Alternatively, the antibody is conjugated to one or more calicheamicin molecules. The calicheamicin family of antibiotics are capable of producing double stranded DNA breaks at sub-picomolar concentrations. Structural analogues of calicheamicin which may be used include, but are not limited to, γ₁¹, α₂¹, α₃¹, N-acetyl-γ₁¹, PSAG or O¹₁ (Hinman et al. Cancer Research 53:3336-3342, 1993; or Lode et al, Cancer Research 58: 2925-2928, 1998).

Enzymatically active toxins and fragments thereof which can be used include, for example, diphtheria A chain, nonbinding active fragments of diphtheria toxin, exotoxin A chain (from Pseudomonas aeruginosa), ricin A chain, abrin A chain, modeccin A chain, alpha sarcin, Aleurites fordii proteins, dianthin proteins, Phytolaca americana proteins (PAPI, PAPII, and PAP-S), momordica charantia inhibitor, curcin, crotin, Asapaonaria officinalis inhibitor, gelonin, mitogellin, restrictocin, phenomycin, eomycin and the tricothecenes. Suitable compounds are described, for example, in WO 93/21232.

The present invention further contemplates an antibody conjugated with a compound with nucleolytic activity (e.g., a ribonuclease or a DNA endonuclease such as a deoxyribonuclease; DNase).

A variety of radioactive isotopes are available for the production of a radioconjugated antibody. Examples include At²¹¹, I¹³¹, I¹²⁵, Y⁹⁰, Re¹⁸⁶, RE¹⁸⁸ Sm¹⁵³, Bi²¹², P³² or radioactive isotopes of Lu.

Conjugates of an antibody and a cytotoxic agent are made using any of a variety of bifunctional protein coupling agents, such as, for example, N-succinimidyl-3-(2-pyriyldithiol) propionate (SPDP), succinimidyl-4-(N-maleimidomethyl)cyclohexane-1-carboxylate, iminothiolane (IT), bifunctional derivatives of imidoesters (such as, dimethyl adipimidate HCL), active esters (such as, disuccinimidyl suberate), aldehydes (such as, glutareldehyde), bis-azido compounds (such as, bis(p-azidobenzoyl)hexanediamine), bis-diazonium derivatives (such as, bis-(p-diazoniumbenzoyl)-ethylenediamine), diisocyanates (such as, tolyene 2,6-diisocyanate), and bis-active fluorine compounds (such as, 1,5-difluoro-2,4-dinitrobenzene). For example, a ricin immunotoxin can be prepared as described in Vitetta et al. Science 238: 1098, 1987. Carbon-14-labeled I isothiocyanatobenzyl-3-methyldiethylene triaminepentaacetic acid (MX-DTPA) is an exemplary chelating agent for conjugation of a radionucleotide to an antibody (e.g., see WO94/11026). The linker may be a “cleavable linker” facilitating release of the cytotoxic drug in the cell. For example, an acid-labile linker, peptidase-sensitive linker, dimethyl linker or disulfide-containing linker (Charm et al. Cancer Research 52:127-131, 1992) may be used.

Alternatively, a fusion protein comprising an antibody and a cytotoxic agent may be made, e.g. by recombinant techniques or peptide synthesis.

In yet another embodiment, the antibody is be conjugated to a “receptor” (such as, streptavidin) for utilization in B cell or antibody producing cell pretargeting wherein the antagonist-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

Suitable methods for determining an antibody for use in the method of the invention will be apparent to the skilled person. For example, a test antibody is applied to a culture of B-ells or a B-cell line and the ability of the antibody to inhibit growth or induce cell death is determined.

For example, methods for isolating and culturing primary B-cells from pigs are described in Kanaan et al., Am J Transplant. 3: 403-15, 2003; from mice in Takahashi et al., J Biotechnol. 49:201-10, 1996; and from humans in Jackson et al., Am J Kidney Dis. 17: 55-61, 1991.

Alternatively, the assay is performed in a B-cell line, such as, for example, a B cell line available from CHS, California, USA.

Such a cell is then contacted with a test antibody for a time and under conditions sufficient for B-cell depletion (e.g., induction of cell death and/or reduction of cell proliferation) to occur and the level of B cell depletion determined. Methods for determining the level of cell growth and/or cell death will be apparent to the skilled person.

For example, APOPTEST (available from Immunotech) stains cells early in apoptosis, and does not require fixation of the cell sample (Martin et al., 1994). This method utilizes an annexin V antibody to detect cell membrane re-configuration that is characteristic of cells undergoing apoptosis. Apoptotic cells stained in this manner can then sorted either by fluorescence activated cell sorting (FACS), ELISA or by adhesion and panning using immobilized annexin V antibodies.

Alternatively, a terminal deoxynucleotidyl transferase-mediated biotinylated UTP nick end-labeling (TUNEL) assay is used to determine the level of cell death. The TUNEL assay uses the enzyme terminal deoxynucleotidyl transferase to label 3′-OH DNA ends, generated during apoptosis, with biotinylated nucleotides. The biotinylated nucleotides are then detected by using streptavidin conjugated to a detectable marker. Kits for TUNEL staining are available from, for example, Intergen Company, Purchase, N.Y.

Alternatively, or in addition, an activated caspase, such as, for example, Caspase 3 is detected. Several caspases are effectors of apoptosis and, as a consequence, are only activated to significant levels in a cell undergoing programmed cell death. Kits for detection of an activated caspase are available from, for example, Promega Corporation, Madison Wis., USA. Such assays are useful for both immunocytochemical or flow cytometric analysis of cell death.

Methods for determining the level of cell proliferation are also known in the art. For example, incorporation of ³H-thymidine or ¹⁴C-thymidine into DNA as it is synthesized is an assay for DNA synthesis associated with cell division. In such an assay, a cell is incubated in the presence of labeled thymidine for a time sufficient for cell division to occur. Following washing to remove any unincorporated thymidine, the label (e.g. the radioactive label) is detected, e.g., using a scintillation counter. Assays for the detection of thymidine incorporation into a live cell are available from, for example, Amersham Pharmacia Biotech.

In another embodiment, cellular proliferation is measured using a MTT assay. The yellow tetrazolium MTT (3-(4,5-dimethylthiazolyl-2)-2,5-diphenyltetrazolium bromide) is reduced by metabolically active cells, in part by the action of dehydrogenase enzymes, to generate reducing equivalents such as NADH and NADPH. The resulting intracellular purple formazan is then solubilized and quantified by spectrophotometric means. Assay kits for MTT assays are available from, for example, American Type Culture Collection.

Alternative assays for determining cellular proliferation, include, for example, measurement of DNA synthesis by BrdU incorporation (by ELISA or immunohistochemistry, kits available from Amersham Pharmacia Biotech), expression of proliferating cell nuclear antigen (PCNA) (by ELISA, FACS or immunohistochemistry, kits available from Oncogen Research Products) or a Hoechst cell proliferation assay that detects DNA synthesis (available from Trevigen Inc.).

A compound that reduces B cell proliferation and/or enhances B cell death is preferred. Preferably, the compound is also tested using other cell types to determine the specificity of the compound for antibody producing cells or B-cells.

A test compound may also be administered to an animal to determine its effect in vivo. In this regard, any animal that produces B cells may be used to determine the efficacy of the compound in depleting B cells. Preferred animals include, for example, a mouse, a rat, a sheep, a pig, a cow or a dog. More preferably, the animal also suffers from a T cell-mediated autoimmune disease, such as, type 1 diabetes or rejection of a pancreatic islet cell graft or rejection of a whole pancreas transplant, thereby enabling assessment of the ability of the compound to treat said disease. For example, the animal is a NOD mouse, a db/db mouse, a BB rat, a PVG rat, a RAG rat or a LEW.1.WR1 rat.

By determining the ability of the compound in depleting B cells in vivo, other parameters, such as, for example, toxicity and/or efficacy and/or specificity of the compound may also be determined. Methods for determining the number of B cells in a sample from an organism are known in the art and/or described herein.

While the assays in the previous paragraphs are described as being useful for determining an antibody that depletes B cells in a subject, these assays are equally applicable to determining any compound that depletes B cells in a subject.

### 2.2 Proteinaceous Compounds

In another embodiment, the compound used to deplete B cells is a proteinaceous compound, such as, for example, a peptide, polypeptide, protein or enzyme. Such a compound acts, for example, by inhibiting production of an antibody producing cell and/or inducing death of an antibody producing cell.

Suitable proteinaceous compounds are known in the art and will be apparent to the skilled person.

For example, the compound is a fusion protein in which a fragment of a BCMA protein is fused to a Fc region of an immunoglobulin. For example, an extracellular domain of a BCMA polypeptide is fused to a Fc region of IgG1. For example, the region of a BCMA polypeptide from amino acid position 2 to about amino acid position 54 is fused to a Fc region of IgG1, preferably, human IgG1. Alternatively, a region of a BCMA polypeptide from about amino acid residue 8 to about amino acid residue 37 is fused to a Fc region of IgG1, for example human IgG1. Alternatively, a region of a BCMA polypeptide from about amino acid residue 8 to about amino acid residue 41 is fused to a Fc region of IgG1, for example human IgG1. Alternatively, a region of a BCMA polypeptide from about amino acid residue 8 to about amino acid residue 88 of a BCMA polypeptide is fused to a Fc region of IgG1, for example human IgG1. The skilled artisan will be aware of suitable sources to determine the amino acid sequence of BCMA, such as, for example, the National Center for Biotechnology Information (NCBI) database available from the National Library of Medicine at the National Institutes of Health of the Government of the United States of America, Bethesda, Md., 20894. In this respect, the amino acid sequence of BCMA is deposited in the NCBI database under the accession number BAB60895 or CAA82690. Alternatively, or in addition, the amino acid sequence of BCMA is provided in WO00/40716 or WO 01/12812. For example, the amino acid sequence of a BCMA polypeptide is set forth in SEQ ID NO: 6.

Methods for the production of such a fusion protein (designated BCMA-Ig or BCMA-Fc) are described in Thompson et al., J.E.M., 192: 129-139, 2000, WO00/40716 and WO 01/12812. Such a fusion protein is also commercially available from Calbiochem or Sigma Aldrich. This fusion protein inhibits the activity of the protein BAFF, which is required for B cell development.

A fusion protein between a fragment of the TACI receptor and a Fc region of an immunoglobulin is also useful for depleting B cells in a subject. For example, Gross et al., Nature, 404: 995-999, 2000 describe the production of TACI-Ig or TACI-Fc comprising a fusion between a fragment of TACI and the Fc region of IgG1. Moreover, WO00/40716 describes the production of TACI-Fc fusion proteins. For example, a region of a TACI polypeptide from about amino acid residue 34 to about amino acid residue 66 is fused to a Fc region of IgG1, for example human IgG1. Alternatively, a region of a TACI polypeptide from about amino acid residue 25 to about amino acid residue 104 is fused to a Fc region of IgG1, for example human IgG1. Alternatively, a region of a TACI polypeptide from about amino acid residue 71 to about amino acid residue 104 is fused to a Fc region of IgG1, for example human IgG1. Alternatively, a region of a TACI polypeptide from about amino acid residue 2 to about amino acid residue 166 is fused to a Fc region of IgG1, for example human IgG1. In this respect, the amino acid sequence of TACI is deposited in the NCBI database under the accession number BAE16555. Alternatively, or in addition, the amino acid sequence of BCMA is provided in WO00/40716. For example, the amino acid sequence of a TACI polypeptide is set forth in SEQ ID NO: 7.

Recombinant TACI-Fc is also commercially available from R and D Systems, Inc., MN, USA.

US Patent Publication No. 20050163775 and International Publication No. WO 02/24909 and Thompson et al., J. Exp. Med. 192: 129-135, 2000, also describe the fusion of a fragment of BR3 or BAFF-R to a Fc region of an immunoglobulin to produce a fusion protein that inhibits B cell production in a subject. For example, a region of a BR-3 or BAFF-R polypeptide from about amino acid residue 2 to about amino acid residue 71 is fused to a Fc region of IgG1, for example human IgG1. In this respect, the amino acid sequence of BR-3 or BAFF-R is deposited in the NCBI database under the accession number BAE16554. Alternatively, or in addition, the amino acid sequence of BR3 or BAFF-R is provided in 20050163775 or WO 02/24909. For example, the amino acid sequence of a BR3 or BAFF-R polypeptide is set forth in SEQ ID NO: 8.

The skilled artisan will be aware of suitable Fc regions to produce a fusion protein as described herein, e.g., BCMA-Ig, TACI-Ig and/or BR3-Ig. For example, the fusion protein comprises an immunoglobulin heavy chain constant region, typically an Fc fragment, which contains two constant region domains and lacks a variable region. Methods for preparing such fusions are disclosed in U.S. Pat. Nos. 5,155,027 and 5,567,584. Such fusions are typically secreted as multimeric molecules wherein the Fc portions are disulfide bonded to each other and two non-Ig polypeptides are arrayed in close proximity to each other.

For example, a suitable Fc region is derived from the Fc region of human IgGi (the hinge region and the CH2 and CH3 domains). This region may be modified so as to remove Fc receptor (FcgRT) and complement (Clq) binding functions.

Alternatively, or in addition the composition comprises a peptide capable of reducing or inhibiting the binding or BAFF to a BAFF receptor, e.g., BCMA, TACI or BR-3. For example, Kayakagi, et al., Immunity 10: 515-524, 2002, showed that the BAFF-binding domain of BR3 resides within a 26-residue core region. Six BR3 residues, when structured within a β-hairpin peptide (bhpBR3), were sufficient to confer BAFF binding and block BR3-mediated signaling. Alternatively, or in addition, the composition comprises an extracellular domain of BCMA or TACI or BR3, capable of binding to BAFF and inhibiting the binding of BAFF to a BAFF receptor.

Alternatively, or in addition a peptide or protein that inhibits B-cell expansion or causes death of a B-cell are selected using a method known in the art. For example, a peptide is produced synthetically. Synthetic peptides are prepared using known techniques of solid phase, liquid phase, or peptide condensation, or any combination thereof, and can include natural and/or unnatural amino acids. Amino acids used for peptide synthesis may be standard Boc (Nα-amino protected Nα-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

Alternatively, a synthetic peptide is produced using a technique known in the art and described, for example, in Stewart and Young (In: Solid Phase Synthesis, Second Edition, Pierce Chemical Co., Rockford, Ill. (1984) and/or Fields and Noble (Int. J. Pept. Protein Res., 35:161-214, 1990), or using an automated synthesizer. Accordingly, peptides of the invention may comprise D-amino acids, a combination of D- and L-amino acids, and various unnatural amino acids (e.g., β-methyl amino acids, Cα-methyl amino acids, and Nα-methyl amino acids, etc) to convey special properties. Synthetic amino acids include ornithine for lysine, fluorophenylalanine for phenylalanine, and norleucine for leucine or isoleucine.

In another embodiment, a peptide is produced using recombinant means. For example, an oligonucleotide or other nucleic acid is placed in operable connection with a promoter. Methods for producing such expression constructs, introducing an expression construct into a cell and expressing and/or purifying the expressed peptide, polypeptide or protein are known in the art and described, for example, in Ausubel et al (In: Current Protocols in Molecular Biology. Wiley Interscience, ISBN 047 150338, 1987); or Sambrook et al (In: Molecular Cloning: Molecular Cloning: A Laboratory Manual, Cold Spring Harbor Laboratories, New York, Third Edition 2001).

Alternatively, a peptide library is screened to identify a compound that for use in the method of the invention. By “peptide library” is meant a plurality of peptides that may be related in sequence and/or structure or unrelated (e.g., random) in their structure and/or sequence. Suitable methods for production of such a library will be apparent to the skilled artisan and/or described herein.

For example, a random peptide library is produced by synthesizing random oligonucleotides of sufficient length to encode a peptide of desired length, e.g., 6 or 9 or 15 amino acids. Methods for the production of an oligonucleotide are known in the art. For example, an oligonucleotide is produced using standard solid-phase phosphoramidite chemistry. Essentially, this method uses protected nucleoside phosphoramidites to produce a short oligonucleotide (i.e., up to about 80 nucleotides). Typically, an initial 5′-protected nucleoside is attached to a polymer resin by its 3′-hydroxy group. The 5′hydroxyl group is then de-protected and the subsequent nucleoside-3′-phosphoramidite in the sequence is then coupled to the de-protected group. The internucleotide bond is then formed by oxidizing the linked nucleosides to form a phosphotriester. By repeating the steps of de-protection, coupling and oxidation an oligonucleotide of desired length and sequence is obtained. Suitable methods of oligonucleotide synthesis are described, for example, in Caruthers, M. H., et al., “Methods in Enzymology,” Vol. 154, pp. 287-314 (1988).

Each of the oligonucleotides is then inserted into an expression construct (in operable connection with a promoter) and introduced into a cell of the invention. Suitable methods for producing a random peptide library are described, for example, in Oldenburg et al., Proc. Natl. Acad. Sci. USA 89:5393-5397, 1992; Valadon et al., J. Mol. Biol., 261:11-22, 1996; Westerink Proc. Natl. Acad. Sci. USA., 92:4021-4025, 1995; or Felici, J. Mol. Biol., 222:301-310, 1991.

### 2.3 Nucleic Acid Compounds

In another embodiment, the compound that depletes B cells from a subject is a nucleic acid based compound, such as, for example, a small interfering RNA (siRNA) compound, a short hairpin RNA (shRNA) compound, an antisense compound, a peptide nucleic acid (PNA) compound, a ribozyme. Preferably, any of these compounds is complementary to or comprises a region that is complementary to and can hybridize to a region of a nucleic acid that encodes a protein that is required for B-cell production and/or development and/or survival. When introduced into a cell using suitable methods, such a nucleic acid inhibits the expression of the target gene encoded by the sense strand.

An anti-sense compound shall be taken to mean an oligonucleotide comprising DNA or RNA or a derivative thereof (e.g., PNA or LNA) that is complementary to at least a portion of a specific nucleic acid target. Preferably, an antisense molecule comprises at least about 15 or 20 or 30 or 40 nucleotides complementary to the nucleotide sequence of a target nucleic acid. The use of antisense methods is known in the art (Marcus-Sakura, Anal. Biochem. 172: 289, 1988).

A ribozyme is an antisense nucleic acid molecule that is capable of specifically binding to and cleaving a target nucleic acid. A ribozyme that binds to a target nucleic acid and cleaves this sequence reduces or inhibits the translation of said nucleic acid. Five different classes of ribozymes have been described based on their nucleotide sequence and/or three dimensional structure, namely, Tetrahymena group I intron, Rnase P, hammerhead ribozymes, hairpin ribozymes and hepatitis delta virus ribozymes.

Generally, a ribozyme comprises a region of nucleotides (e.g., about 12 to 15 nucleotides) that are complementary to a target sequence.

An RNAi (or siRNA or small interfering RNA) is a double stranded RNA molecule that is identical to a specific gene product. The dsRNA when expressed or introduced into a cell induces expression of a pathway that results in specific cleavage of a nucleic acid highly homologous to the dsRNA.

RNAi molecules are described, for example, by Fire et al., Nature 391: 806-811, 1998, and reviewed by Sharp, Genes and Development, 13: 139-141, 1999). As will be known to those skilled in the art, short hairpin RNA (“shRNA”) is similar to siRNA. However, the shRNA molecule comprises a single strand of nucleic acid with two complementary regions (highly homologous to the sequence of a region of an IRES or the complement thereof) separated by an intervening hairpin loop such that, following introduction to a cell, it is processed by cleavage of the hairpin loop into siRNA.

A preferred siRNA or shRNA molecule comprises a nucleotide sequence that is identical to about 19-21 contiguous nucleotides of the target mRNA. Preferably, the target sequence commences with the dinucleotide AA, comprises a GC-content of about 30-70% (preferably, 30-60%, more preferably 40-60% and more preferably about 45%-55%), and is specific to the nucleic acid of interest.

Suitable RNAi or shRNA molecules and vectors comprising same for the inhibition of BAFF expression and thereby B cell production are described, for example, in International Application No. PCT/AU2004/000215.

### 2.5 Small Molecules

In another embodiment, the compound is a small molecule. The structure of small molecules varies considerably as do methods for their synthesis. The present invention contemplates the screening of any small molecule or small molecule library to identify a compound capable of depleting antibody producing cells in a subject.

An example of a suitable small molecule library is described, for example, in U.S. Pat. No. 6,168,192. The method described is for producing a multidimensional chemical library that comprises converting a set of at least two different α-allyl carbonyl monomers to form monomer derivatives by converting the allyl group to another group and covalently linking at least two of the produced monomers to form oligomers.

In an alternative method, McMillan et al., (Proc Natl Acad Sci USA. 97: 1506-1511, 2000) describes the production of an encoded chemical library (ECLiPS method) based on a pyrimidineimidazole core prepared on polyethylene glycol-grafted polystyrene support. Compounds are attached to resin by a photolabile o-nitrobenzyl amide linker. The first synthetic step introduced primary amines. After a pool and split step, the amines are acylated with fluorenylmethoxycarbonyl (Fmoc)-protected amino acids. A second pool and split step is performed, followed by Fmoc deprotection and subsequent heteroarylation of the resulting free amines by electrophilic substitution with a set of nine substituted pyrimidines. The library produced comprised 8,649 compounds.

Additional small molecule libraries include, for example, libraries comprising statine esters (U.S. Pat. No. 6,255,120), neomycin analogs (U.S. Pat. No. 6,207,820), fused 2, 4-pyrimidinediones (U.S. Pat. No. 6,025,371), dihydrobenzopyran based molecules (U.S. Pat. No. 6,017,768), 1,4-benzodiazepin-2,5-dione based compounds (U.S. Pat. No. 5,962,337), benmzofuran derivatives (U.S. Pat. No. 5,919,955), indole derivatives (U.S. Pat. No. 5,856,496), products of polyketides (U.S. Pat. No. 5,712,146), morpholino compounds (U.S. Pat. No. 5,698,685) or sulphonamide compounds (U.S. Pat. No. 5,618,825).

Compounds screened to determine their ability to deplete antibody producing cells in a subject can be screened using high throughput screening techniques, such as, for example, sequential high throughput screening (SHTS). SHTS is an iterative process of screening a sample of compounds for activity, analyzing the results, and selecting a new set of compounds for screening, based on compounds identified in one or more previous screens. Selection of compounds is driven by finding structure activity relationships (SARs) within the screened compounds and using those relationships to drive further selection.

Recursive partitioning (RP) is a statistical methodology that can be used in conjunction with high-throughput screening techniques, such as, SHTS, by identifying relationships between specific chemical structural features of the molecules and biological activity. The premise of this method is that the biological activity of a compound is a consequence of its molecular structure. Accordingly, it is useful to identify those aspects of molecular structure that are relevant to a particular biological activity. By gaining a better understanding of the mechanism by which the compound acts, additional compounds for screening can more accurately be selected. Suitable RP methods are described, for example in Hawkins, D. M. and Kass, G. V., (In: Automatic Interaction Detection. In Topics in Applied Multivariate Analysis; Hawkins, D. H., Ed.; 1982, Cambridge University Press, pp. 269-302).

Quantitative structure activity relationship (QSAR) is also useful for determining a feature or features of a compound required for or useful for a desired biological activity. QSAR models are determined using sets of compounds whose molecular structure and biological activity are known, a training set. QSAR approaches are either linear or nonlinear. The linear approach assumes that the activity varies linearly with the level of whatever features affect it, and that there are no interactions among the different features.

Nonlinear QSAR approaches account for the fact that activity can result from threshold effects; a feature must be present for at least some threshold level for activity to occur. Furthermore, as interactions between features are observed in many QSAR settings, the utility of one feature depends upon the presence of another. For example, activity may require the simultaneous presence of two features.

### 2.5 Combination Therapies

The present invention also contemplates the use of a plurality of compounds to deplete B cells from a subject. Such compounds may be administered in a single formulation or separately.

For example, US Patent Application No. 20050163775 describes a combination therapy that comprises an anti-CD-20 antibody (e.g., as described supra) and a BAFF antagonist (e.g., as described supra, e.g., a BR3 fragment) that is useful for the depletion of B cells in a subject.

US Patent Application No. 20050123540 describes a combination therapy comprising an antibody that binds to CD-19, CD-20, CD-22 or CD-37 and an antibody that binds to an immunoregulatory molecule (e.g., B-7, CD-23 or CD-40) to deplete B-cells from a subject.

Alternatively, or in addition, a compound as described supra (e.g., rituximab) is combined with a compound, such as, for example, prednisolone and/or cyclophosphamide and/or interleukin (IL)-21 to enhance B-cell depletion in a subject (van Vollenhoven et al., Scand J Rheumatol. 33: 423-7, 2004).

### 2.6 Pharmaceutical Formulations

As the compound that depletes antibody producing cells is administered to a subject, it is preferred that it is produced as a pharmaceutical formulation. In this regard, the present invention additionally contemplates administering a formulation or composition comprising the compound to a subject in the method of treatment of the invention.

To prepare pharmaceutical or sterile compositions including a compound that depletes B cells in a subject, the compound is mixed with a pharmaceutically acceptable carrier or excipient. Formulations of a therapeutic compound are prepared, for example, by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic formulation depends on several factors, including the serum or tissue turnover rate of the entity, the level of symptoms, the immunogenicity of the entity, and the accessibility of the target cells in the biological matrix. Preferably, an administration regimen maximizes the amount of therapeutic compound delivered to the patient consistent with an acceptable level of side effects. Accordingly, the amount of formulation delivered depends in part on the particular entity and the severity of the condition being treated. Guidance in selecting appropriate doses of antibodies, cytokines, and small molecules are available (see, e.g., Wawrzynozak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. New Engl. J. Med. 348:601-608, 2003; Milgrom, et al. New Engl. J. Med. 341:1966-1973, 1999; Slamon, et al. New Engl. J. Med. 344:783-792, 2001; Beniaminovitz, et al. New Engl. J. Med. 342:613-619, 2000; Ghosh, et al. New Engl. J. Med. 348:24-32, 2003; or Lipsky, et al. New Engl. J. Med. 343:1594-1602, 2000).

An antibody, antibody fragment, or other proteinaceous compound is provided, for example, by continuous infusion, or by doses at intervals of, e.g., one day, one week, or 1-7 times per week. Doses of a formulation may be provided intravenously, subcutaneously, topically, orally, nasally, rectally, intramuscular, intracerebrally, or by inhalation. A preferred dose protocol is one involving the maximal dose or dose frequency that avoids significant undesirable side effects. A total weekly dose depends on the type and activity of the compound being used to deplete B cells. For example, such a dose is at least about 0.05 μg/kg body weight, or at least about 0.2 μg/kg, or at least about 0.5 μg/kg, or at least about 1 μg/kg, or at least about 10 μg/kg, or at least about 100 μg/kg, or at least about 0.2 mg/kg, or at least about 1.0 mg/kg, or at least about 2.0 mg/kg, or at least about 10 mg/kg, or at least about 25 mg/kg, or at least about 50 mg/kg (see, e.g., Yang, et al. New Engl. J. Med 349:427-434, 2003; or Herold, et al. New Engl. J. Med. 346:1692-1698, 2002.

For example, BCMA-Ig and/or TACI-Ig and/or BR3-Ig is/are administered BR3-Fc at a dosage range of 0.5 mg/kg to 10 mg/kg body weight, preferably 1 mg/kg to 5 mg/kg, more preferably, 1.5 mg/kg to 2.5 mg/kg. In one embodiment, BCMA-Ig and/or TACI-Ig and/or BR3-Ig is/are administered at 5 mg/kg every other day from day 1 to day 12 of treatment. Also contemplated is dosing at about 2-5 mg/kg every 2-3 days for a total of 2-5 doses.

For example, TACI-Ig is administered to a subject by intravenous injection at a concentration of 2 mg/kg, 5 mg/kg, 7 mg/kg or 10 mg/kg on a weekly basis for at least about 5 weeks, e.g., for at least about 7 weeks or more, e.g., for at least about 10 weeks or more.

In the case of an antibody, e.g., a chimeric antibody (e.g., Rituxan), such an antibody is administered at a dosage, such as, for example, 500 mg per dose every other week for a total of 2 doses. A humanized anti-CD20 antibody is administered, for example, at less than 500 mg per dose such as at between about 200-500 mg per dose, between about 250 mg-450 mg, or 300-400 mg per dose, for 2-4 doses every other week or every third week.

The desired dose of a small molecule therapeutic, e.g., natural product, or organic chemical, or a peptide is about the same as for an antibody or polypeptide, on a moles/kg body weight basis. The desired plasma concentration of a small molecule therapeutic or peptide is about the same as for an antibody, on a moles/kg body weight basis.

An effective amount of a compound for a particular patient may vary depending on factors such as the condition being treated, the overall health of the patient, the method route and dose of administration and the severity of side affects, see, e.g., Maynard, et al. (1996) A Handbook of SOPs for Good Clinical Practice, Interpharm Press, Boca Raton, Fla.; or Dent (2001) Good Laboratory and Good Clinical Practice, Urch Publ., London, UK.

Determination of the appropriate dose is made by a clinician, e.g., using parameters or factors known or suspected in the art to affect treatment or predicted to affect treatment. Generally, the dose begins with an amount somewhat less than the optimum dose and is increased by small increments thereafter until the desired or optimum effect is achieved relative to any negative side effects. Important diagnostic measures include those of symptoms of, e.g., the level of B cell expansion or antibodies produced against a marker of a T cell-mediated autoimmune disease, such as, type 1 diabetes or rejection of a pancreatic islet cell graft or rejection of a whole pancreas transplant. Preferably, a compound that will be used is derived from or adapted for use in the same species as the subject targeted for treatment, thereby minimizing a humoral response to the reagent.

An effective amount of therapeutic will decrease disease symptoms, for example, as described supra, typically by at least about 10%; usually by at least about 20%; preferably at least about 30%; more preferably at least about 40%, and more preferably by at least about 50%.

The route of administration is by, e.g., topical or cutaneous application, injection or infusion by intravenous, intraperitoneal, intracerebral, intramuscular, intraocular, intraarterial, intracerebrospinal, intralesional, or pulmonary routes, or by sustained release systems or an implant (see, e.g., Sidman et al. Biopolymers 22:547-556, 1983; Langer, et al. J. Biomed. Mater. Res. 15:167-277, 1981; Langer Chem. Tech. 12:98-105, 1982; Epstein, et al. Proc. Natl. Acad. Sci. USA 82:3688-3692, 1985; Hwang, et al. Proc. Natl. Acad. Sci. USA 77:4030-4034, 1980; U.S. Pat. Nos. 6,350,466 and 6,316,024).

The route of administration generally depends upon the type of compound used. For example, rituximab is administered to a human subject by weekly injection and/or infusion (e.g., Rouzière et al., Arthritis Res. and Ther., 7: 714-724, 2005).

### 3. Timing of Administration

Preferably, the compound that depletes or reduces antibody producing cell numbers in a subject is administered prior to or concomitant with an immune response by the subject against a pancreatic β-islet cell. In this manner the number of antibody producing cells is reduced prior to or during the immune response against the β-islet cell/s, thereby reducing or preventing said immune response and preventing the onset of a T cell-mediated autoimmune disease, such as, type 1 diabetes or rejection of a pancreatic islet cell graft or rejection of a whole pancreas transplant or reducing the severity of the a T cell-mediated autoimmune disease, such as, type 1 diabetes or rejection of a pancreatic islet cell graft or rejection of a whole pancreas transplant.

In a preferred embodiment, the compound is administered immediately prior to or concomitant with the onset of an immune response against a pancreatic β-islet cell in the subject being treated. Accordingly, in one embodiment, the method of the invention comprises determining or predicting the onset of the immune response against a pancreatic β-islet cell in the subject.

Methods for determining or predicting the onset of the immune response will be apparent to the skilled person and/or described herein.

For example, the detection of an auto-antibody against an antigen derived from or on the surface of a pancreatic β-cell is indicative of an immune response against said cell by a subject.

One such assay detects islet cell antibodies in the serum of a subject. This assay comprises contacting a section of a pancreas comprising an islet cell with serum from a test subject. Immunoglobulin in the serum from the subject that is capable of binding to a pancreatic β-islet cell is then detected using a secondary labeled antibody that binds to human immunoglobulin. The label bound to the antibody is detected using microscopy, and labeling of antibodies bound to a pancreatic β-islet cell is indicative of pancreatic cancer.

Methods of detection used in such an assay will depend on the label used with the secondary antibody. For example, the antibody may be labeled with an enzyme that requires addition of a substrate to facilitate detection, e.g., alkaline phosphatase, β-galactosidase or horseradish peroxidase. Alternatively, the secondary antibody is labeled with a fluorescent label, e.g., FITC, Texas Red or a fluorescent nanocrystal, and the antibody is detected by exposing the pancreatic tissue to light of a suitable wavelength to excite the fluorescent label. A suitable method for detecting islet cell antibodies using a fluorescent marker is described, for example, in Bottazzo et al., Lancet 2: 1279-83, 1974.

Alternatively, or in addition, an assay is used to detect an auto-antibody that binds to a specific antigen in a subject. A suitable antigen will be apparent to the skilled person, for example, a suitable antigen is selected from the group consisting of glutamic acid decarboxylase (GAD) (Solimena et al., N Engl J Med 318:1012-1020, 1988), ICA512 (IA-2) (Rabin et al., Diabetes. 41:183-6, 1992), IA-2β (phogrin) (Rabin et al., supra), insulin (Palmer et al, Science. 222:1337-9, 1983), proinsulin, preproinnsulin (Moriyama et al. Proc Natl Acad Sci USA, 100:10376-10381, 2003), insulin beta chain [amino acids 9-23], insulin alpha chain [amino acids 1-15], glycolipid autoantigen bound by monoclonal antibody A2B5 (Kundu et al., Biochem Biophys Res Commun. 116:836-842, 1983), glycolipid autoantigen bound by monoclonal antibody 3G5 (Nayak et al., Kidney Int. 41:1638-1645, 1992), glycolipid autoantigen bound by monoclonal antibody R2D6 (Alejandro et al., J Clin Invest. 74:25-38, 1984), ICA69 (Stassi et al., Diabetol, 40:120-121, 1997), carboxypeptidase H, ICA12 (SOX13).

Methods for the detection of auto-antibodies will be apparent to the skilled person. For example, an immunoassay, such as, for example, an ELISA or a FLISA or a RIA.

Methods for performing an ELISA or a FLISA for detecting an antibody in a sample will be apparent to the skilled person. For example, an antigen (e.g., an autoantigen described herein or an epitope thereof) is immobilized on a solid support, such as, for example, a glass plate or a microtitre plate well. A sample derived from a subject (e.g., a serum sample or a plasma sample) is then contacted tot eh antibody for a time and under conditions sufficient for an antibody/antigen complex to form. The complex is then detected by contacting the immobilized complex with an anti-human Ig (e.g., anti-human IgG) antibody (assuming that the test sample is from a human). Preferably, the detecting antibody is labeled with a detectable marker. Alternatively, an additional antibody or ligand capable of binding to the detecting antibody is used that is labeled with a detectable marker.

In the case of an ELISA, the detecting antibody is preferably labeled with an enzyme, e.g., horseradish peroxidase or alkaline phosphatase. A substrate of the enzymatic label that is converted to a detectable compound in the presence of the label is then added, and the level of detectable compound determined. The level of detectable compound that is detected is indicative of the amount of antibody against the auto-antigen in the biological sample.

A FLISA is similar to an ELISA, however, the primary or secondary antibody or tertiary antibody/molecule is labeled with a fluorescent label (for example, a Texas Red Label or a FITC label) or a fluorescent nanocrystal (for example as disclosed in U.S. Pat. No. 6,306,610 or available from Qdot™, Hayward, Calif.). Such a fluorescent label is directly detectable, rather than requiring a substrate. Methods of detecting a fluorescent label will be apparent to the skilled artisan.

By way of example, Brooking et al. (Clin Chim Acta 331:55-59, 2003) describe an ELISA based assay for the detection of auto-antibodies against GAD65. The described assay uses a low concentration of the GAD antigen on a microtitre plate to capture the auto-antibodies in a sample. Biotinylated GAD in the fluid phase is added and is captured by the second binding site of the autoantibody, and it is the biotinylated GAD65 that is detected to produce a non-isotopic detectable signal

Alternatively, the presence of an antibody that binds to an autoantigen is detected using a radioimmunoassay (RIA). The basic principle of the assay is the use of a radiolabeled antigen to detect antibody-antigen interactions. An antibody in a test sample is bound to or immobilized on a solid support (or the assay may be performed in the liquid phase) and a sample brought into direct contact with said antibody. To detect the level of bound antigen, an isolated and/or recombinant form of the antigen is radiolabeled and brought into contact with the same antibody. Following washing, the level of bound radioactivity is detected. As any antigen in the biological sample inhibits binding of the radiolabeled antigen the level of radioactivity detected is inversely proportional to the level of antigen in the sample. Such an assay may be quantitated by using a standard curve using increasing known concentrations of the isolated antigen.

Another form of immunoassay (a fluid phase assay) used to detect auto-antibodies involves incubating labeled antigen (e.g., radioactively labeled) with patient sera and placing samples in 96-well filtration plates, where a “bead” (e.g. Sepharose) with coupled protein A and/or protein G is added. Free radioactivity (i.e., unbound antigen) is then removed by filtration washing. Scintillation fluid is added directly to the 96-well filtration plates, and counting is performed on multichannel beta counters. This form of assay has been used to detect GAD65, ICA512 and IA-2 (Falorni et al., J Immunol Methods 186:89-99, 1995; and Kawasaki et al., Frontiers in Bioscience 5:181-190, 2000).

Nagata et al., Ann. New York Acad. Sci. 1037: 10-15, 2004 describe an ELISPOT assay useful for detecting the presence of auto-antibodies against insulin, IA-2 and GAD65.

The present inventors have found that the number of B cells expand at about the time of the immune response against the β-islet cell/s. Accordingly, in a preferred embodiment, the compound is administered at the time of or immediately prior to B cell expansion in a subject. Methods for determining B cell expansion in a subject will be apparent to the skilled person.

For example, the number of B cells is determined in samples obtained temporally from a subject. Preferably, the sample obtained from the subject is of a constant volume. The number of B cells is then determined, for example, using FACS analysis or immunohistochemistry or by isolating B cells using, for example a magnetic cell sorter each of which make use of an antibody or ligand capable of binding to a B cell marker (preferably, a surface expressed B cell marker). A suitable B cell marker will be apparent to the skilled artisan and includes, for example, CD-20, CD-19, CD-22, B220.

Such a screening method additionally enables the detection and/or quantification of a specific class or type of B cell. For example, mature and immature B cells are detected by detecting a cell expressing CD45R, CD4, IgM and IgD while MZB cells are detected by detecting a cell expressing CD45, CD4, CD21/35, CD23 and/or CD1d.

In a preferred embodiment, the level of expansion of a specific type of B cell is determined. Preferably, the level of expansion of the B cell type is determined relative to another cell type (e.g., B cell type) that does not expand in a T cell-mediated autoimmune disease, such as, type 1 diabetes or rejection of a pancreatic islet cell graft or rejection of a whole pancreas transplant. For example, the level or number of MZB cells is determined in a subject relative to the number of a constant cell type and this number compared to the relative level of MZB cells compared to the constant cell type in a suitable reference sample. An increase in the relative level of MZB cells in the test sample relative to the reference sample is indicative of the onset of an immune response against a β-islet cell and/or B cell expansion and that the subject is in need of treatment using the method of the invention.

In another embodiment, the expansion of B cells or specific B cells is determined in a biopsy derived from the pancreas of a subject at risk of developing diabetes. Preferably, the number of MZB cells is determined in such a biopsy. Subjects that have increased levels of B cells infiltrating the pancreatic islet are considered to be raising an immune response against a β-islet cell and suitable for treatment using the method of the invention. Suitable methods for the detection of B cells in a biopsy will be apparent tot eh skilled person, and include, for example, immunohistochemistry and/or immunofluorescence.

For example, a cell or tissue section (e.g., a biopsy sample) that is to be analyzed to determine the level of a B cell marker is fixed to stabilize and protect both the cell and the proteins contained within the cell. Preferably, the method of fixation does not disrupt or destroy the antigenicity of the B cell marker. Methods of fixing a cell are known in the art and include for example, treatment with paraformaldehyde, treatment with alcohol, treatment with acetone, treatment with methanol, treatment with Bouin's fixative and treatment with glutaraldehyde. Following fixation a cell is incubated with a ligand or antibody capable of binding to the B cell marker (e.g., as described supra). The ligand or antibody is, for example, labeled with a detectable marker, such as, for example, a fluorescent label (e.g. FITC or Texas Red), a fluorescent semiconductor nanocrystal (as described in U.S. Pat. No. 6,306,610) or an enzyme (e.g. horseradish peroxidase (HRP)), alkaline phosphatase (AP) or β-galactosidase. Alternatively, a second labeled antibody that binds to the first antibody is used to detect the first antibody. Following washing to remove any unbound antibody, the level of the protein bound to said labeled antibody is detected using the relevant detection means. Means for detecting a fluorescent label will vary depending upon the type of label used and will be apparent to the skilled artisan. By determining the number of B cells (or specific B cells) in a biopsy, or in a pancreatic islet in the sample a diagnosis of the onset of a T cell-mediated autoimmune disease, such as, type 1 diabetes or rejection of a pancreatic islet cell graft or rejection of a whole pancreas transplant is made). More detailed methods of immunohistochemistry and/or immunofluorescence are described in, for example, Immunohistochemistry (Cuello, 1984 John Wiley and Sons, ASIN 0471900524).

As will be apparent to the skilled person from the foregoing, in one embodiment of the invention, a method for determining the level of B cell expansion comprises:
(i) determining the level of B cells or a type of B cell in a sample derived from a subject suspected of suffering from or at risk of suffering from a T cell-mediated autoimmune disease, such as, type 1 diabetes or rejection of a pancreatic islet cell graft or rejection of a whole pancreas transplant; and
(ii) determining the level of B cells or a type of B cell in a suitable reference sample,
   wherein an increased level of the B cells or the type of B cell at (i) compared to (ii) indicates that the B cells are expanding in the subject. Furthermore, such a result indicates that a subject should be treated using the method of treatment of the invention.

In another embodiment, B cell expansion or proliferation is determined by detecting the level of a molecule that is associated with B cell development.

As used herein, the term “molecule that is associated with B cell development” shall be take to include a peptide, polypeptide or protein that causes, enhances or inhibits the level of B cell proliferation, differentiation or cell death in a subject. Suitable molecules include, for example, BAFF polypeptide, TACI polypeptide or BCMA polypeptide. Suitable methods for determining the level of such a molecule are known in the art and/or described herein. For example, an ELISA is used to determine the level of a molecule that is associated with B cell development in a serum sample from a suitable control sample. For example, an ELISA kit for detecting the level of BAFF in a sample is commercially available from Bender MedSystems, Vienna, Austria.

As will be apparent to the skilled artisan from the preceding paragraph, in one embodiment, the level of B cell or antibody producing cell expansion in a subject is determined by performing a method comprising:
(i) determining the level of a molecule that is associated with B cell development in a sample derived from a test subject; and
(ii) determining the level of the molecule that is associated with B cell development in a suitable reference sample,
   wherein an increased level of the molecule at (i) compared to (ii) is indicative of expansion of antibody producing cells and/or B cells in the subject, such a result is also indicative of a subject suitable for treatment using the method of the invention.

Alternatively, or in addition, the onset of an immune response against a pancreatic P islet cell is determined by detecting in a sample from a subject a T cell that binds to or is capable of binding to a pancreatic β islet cell antigen, e.g., a protein expressed on the surface of a pancreatic β islet cell. For example, the method comprises detecting in a sample from a subject a T cell capable of binding to an islet-specific glucose-6-phosphatase-related protein (IGRP) or an immunogenic fragment or epitope thereof. For example, the method comprises contacting a T cell containing fraction from a subject with a protein complex (e.g., a tetramer) comprising an islet-specific glucose-6-phosphatase-related protein (IGRP) or an immunogenic fragment or epitope thereof and detecting a T cell bound to said protein complex (e.g., tetramer), wherein detection of the T cell bound to the protein, fragment, epitope or complex is indicative of the onset of an immune response against a pancreatic β-islet cell by the subject. Suitable methods for detecting such a tetramer are described, for example, in Lieberman et al., Proc Natl Acad Sci USA. 100: 8384-8, 2003 or Yang et al., J Immunol. 176: 2781-9, 2006.

A method for determining the timing of administration of a compound as described herein according to any embodiment may be performed with a sample isolated previously from a subject. Accordingly, the method is performed ex vivo.

It will be apparent from the preceding description that a method for determining the onset of an immune response by a subject against a pancreatic β cell provided by the present invention may involve a degree of quantification. Such quantification is readily provided by the inclusion of appropriate reference samples in the assays as described below.

As will be apparent to the skilled artisan, when internal controls are not included in each assay conducted, the control may be derived from an established data set.

Suitable reference sample include, for example, a reference sample is selected from the group consisting of:
(i) a sample from a normal subject;
(ii) a sample from a healthy subject;
(iii) an extract of (i) or (ii);
(iv) a fraction of (i) or (ii);
(v) a data set comprising measurements of the number of B cells in a sample from a healthy individual or a population of normal individuals;
(vi) a data set comprising measurements of the number of B cells in a sample from a normal individual or a population of normal individuals; and

For example, the reference sample is (i) or (ii) described above.

Those skilled in the art are readily capable of determining the baseline for comparison in any diagnostic assay of the present invention without undue experimentation, based upon the teaching provided herein.

In the present context, the term “healthy individual” shall be taken to mean an individual who is known not to suffer from diabetes, such knowledge being derived from clinical data on the individual. It is preferred that the healthy individual is asymptomatic with respect to the any symptoms associated with diabetes.

The term “normal individual” shall be taken to mean an individual that has not developed auto-antibodies to a pancreatic β-cell marker and/or having a normal number of B cells as described herein in a particular sample derived from said individual.

As will be known to those skilled in the art, data obtained from a sufficiently large sample of the population will normalize, allowing the generation of a data set for determining the average level of a particular parameter. Accordingly, the B cells as described herein can be determined for any population of individuals, and for any sample derived from said individual, for subsequent comparison to levels determined for a sample being assayed. Where such normalized data sets are relied upon, internal controls are preferably included in each assay conducted to control for variation.

The present invention also contemplates treating a subject at risk of developing a T cell-mediated autoimmune disease, such as, type 1 diabetes or rejection of a pancreatic islet cell graft or rejection of a whole pancreas transplant (e.g., as described supra) using a method described herein according to any embodiment during the period of life in which the majority of subjects in a population develop a T cell-mediated autoimmune disease, such as, type 1 diabetes or rejection of a pancreatic islet cell graft or rejection of a whole pancreas transplant. For example, a subject at risk of developing type 1 diabetes is treated using the method of the invention between about 4 years of age and about 6 years of age and/or between about 10 years of age and about 14 years of age.

Preferably, a subject at risk of developing a T cell-mediated autoimmune disease, such as, type 1 diabetes or rejection of a pancreatic islet cell graft or rejection of a whole pancreas transplant is monitored using a method described herein to determine onset of an immune response, e.g., against a β-islet cell and/or B cell proliferation and/or B cell infiltrating their pancreas to thereby determine a subject suitable for treatment using the method of the invention.

By timing the treatment such that it is administered at the time of or immediately prior to B cell expansion and/or an immune response against a β-islet cell and/or at the time of an increase in serum glucose levels the inventors provide increased protection for a subject against the development of a T cell-mediated autoimmune disease, such as, type 1 diabetes or rejection of a pancreatic islet cell graft or rejection of a whole pancreas transplant while also enabling the B cells to recover following depletion. Accordingly, following a suitable time, the subject will regain their normal complement of B cells without those that produce antibodies that recognize or bind to β-islet cells and/or induce an immune response against said cells.

The present invention is described further in the following non-limiting examples.

### Example 1

### Perturbed B Lymphocyte Compartment in NOD Mice

### 1.1 Methods

Mice. C57BL/6, DBA, BALB/c, NOD.SCID and NOD/Lt (NOD) mice were obtained form WEHI Kew, Melbourne, Australia.

Detection of diabetes. Diabetes was determined by measurement of blood glucose levels (BGL) using an Accu-Check Advantage glucometer with Accu-Check II strips (Roche). Mice were monitored twice-weekly from 10 weeks-of-age onwards, mice with a BGL>18.0 nmol/L on 2 consecutive readings were considered diabetic.

Flow cytometry. Lymphocytes were isolated from spleen, pancreatic lymph nodes (PLN) and pancreas using standard techniques. Primary biotin-FITC-, PE, PerCP and APC-labeled monoclonal rat antibodies against mouse cell surface antigens B220/CD45R (RA-6B2), CD4 (L3T4)(GK1.5), CD8a (Ly2)(53-6-7), IgM (11/41), CD21/CD35 (CR2/CR1, CD23 (FcεRII)(B3B4), CD1d (CD1.1, Ly-3B)(1B1) and CD9 (KMC8), CD40 (3/23), BAFFR (B2G1), as well as secondary reagents were purchased from BD Biosciences, San Jose, Calif. BAFF binding was assessed by staining with an BAFF-IgG₂ₐ/Fc chimeric construct and detected with an anti-IgG2a-biotin antibody. Flow cytometric analysis was conducted on a FACScalibur flow cytometer (BD Biosciences). Mature B-lymphocyte and transitional subpopulations were identified based on the expression pattern of the surface markers IgM, B220, CD21 and CD23 essentially as described in Loder et al., J Exp Med 190:75-89, 1999.

Histology. Pancreata were snap frozen and stained with Hematoxylin-and-eosin using standard techniques and analyzed for islet morphology and degree of insulitis using standard methods. Expansion of the splenic marginal zone was analyzed using anti-mouse CD1d-biotin (BD Biosciences) and rat anti-mouse Moma-1 which identifies metalophillic macrophages (Serotec/Australia Laboratory Services Pty Ltd). Primary antibody labeling was revealed with HRP-linked anti-rat IgG and alkaline phosphatase (AP)-linked streptavidin. Chromogenic substrate reagents diaminobenzidine (DAB; Sigma) and NBT/BCIP (Sigma) were used to develop HRP and AP, respectively. All immunohistochemistry slides were observed under a Leica light microscope and images were captured using a Leica DC 200 camera (Leica).

### 1.2 Results

NOD mice develop spontaneous autoimmune diabetes making them an excellent model for research, as well as an important model for pre-clinical testing of novel therapeutics (Delovitch, et al., Immunity 7:727, 1997 (published erratum appears in Immunity 4:531, 1998)).

To understand the role of B lymphocytes in diabetes development B cell subsets, B cell function and kinetics were assayed with relation to age, location and stage of disease.

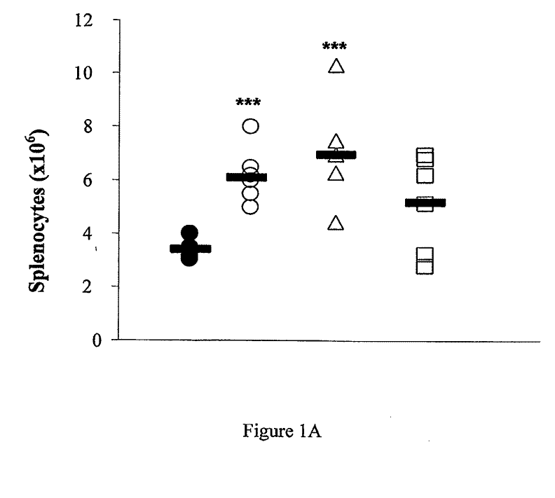

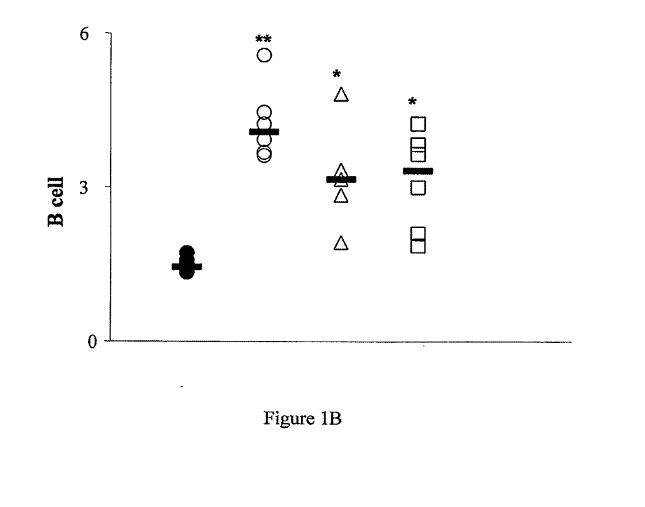

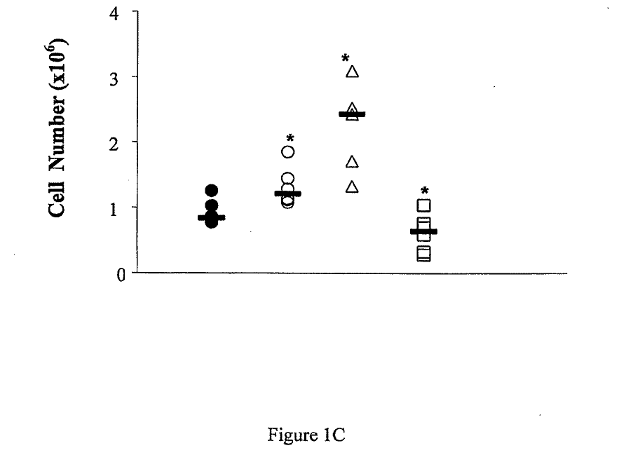

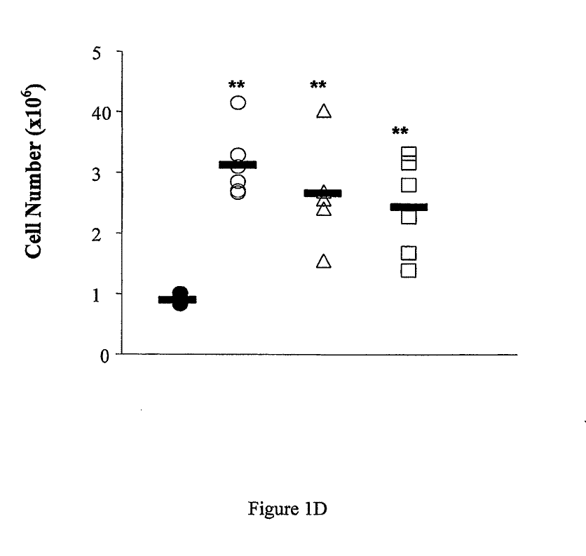

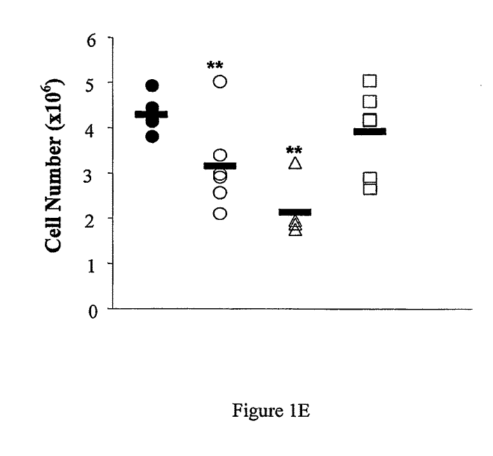

In this respect, the peripheral B lymphocyte compartment of young female NOD mice was analyzed. Young mice were selected as later autoimmunity may mask underlying alterations in the immune system that might pre-dispose NOD mice to diabetes. Analysis of lymphocyte numbers in the spleen revealed that female NOD mice have about 30-50% fewer splenocytes and are thus relatively lymphopenic compared to non-diabetic mouse strains (FIG. 1A). The decrease in splenic cell numbers related to an absolute decrease (≧60%) in the numbers of B lymphocytes, whereas T cell numbers fell within ranges seen for non-diabetic strains of mice (FIGS. 1B and 1C).

The distribution and numbers of B lymphocyte subsets based upon the differential expression of B220, IgM, CD23 and CD21 was then analyzed. Analysis of mature B lymphocyte subsets revealed a decreased number of follicular (FoB) B lymphocytes in the blood and spleen (FIG. 1D). In contrast, NOD mice exhibited markedly expanded numbers of marginal zone (MZB) B lymphocytes, resulting in a decreased ratio of FoB:MZB, from ˜10:1 in non-diabetic strains to ˜4:1 in NOD mice (FIG. 1E).

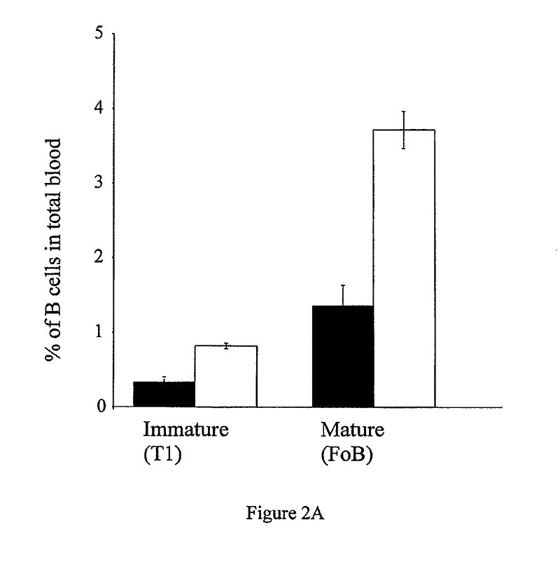

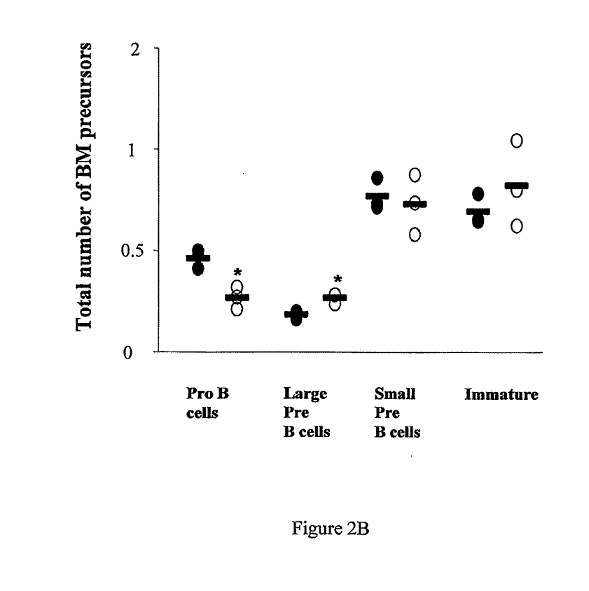

Though mature B cell percentages in the blood were within normal ranges, the numbers of transitional type 1 (T1) B lymphocyte precursors were reduced (FIG. 2A), suggesting a defect in the bone marrow effecting B cell hematopoiesis. However, analysis of the numbers of B lymphocyte bone-marrow precursors did not reveal any differences to control strains (FIG. 2B), indicating that B lymphocyte survival, or transit through the blood may be impaired in NOD mice.

### Example 2

### Factors Affecting the Expanded MZB Compartment in NOD Mice

### 2.1 Methods

Lymphocyte purification. Enriched total T- and B-lymphocytes were obtained by magnetic separation using murine MACS Pan-T-cell or B-cell isolation kits respectively (Miltenyi Biotec, Sydney, Australia). Purities of >97% were obtained. B-cell subpopulations were further purified by FACS based upon the staining pattern obtained with B220, IgM, CD21 and CD23 monoclonal antibodies. Pure (>98%) subpopulations were obtained using a FACSdiva instrument (BD Biosciences).

In vitro B-cell stimulation assays. Purified mature B-cells were seeded at 1×10⁵ per well into round-bottom microtitre plates in 100 μl medium (RPMI1650; Gibco/Invitrogen, 10% heat-inactivated FCS; Gibco Life Technologies, 1:100 penicillin/Streptomycin; Gibco Life Technologies, 50 μM 2-ME; Merck) and cultured in triplicate with either the F(ab)₂-fragment of goat anti-murine IgM (μ-chain specific, 20 μg/ml; Jackson Immunoresearch), IL-4 (100 ng/ml; Gibco/Invitrogen), anti-mouse CD40 (HM40-3)(1 μg/ml; BD Biosciences), LPS (500 mg/ml; Gibco/Invitrogen) or bacterial DNA (CpG) (3 μg/ml; Gibco/Invitrogen) for 48 h. Cells were pulsed for the last 24 h with ³[H]-thymidine (1 μCi/well; Amersham Biosciences), harvested and then assayed for ³[H]-thymidine incorporation (cpm).

Chemotaxis assays. Responses to Sphingosine 1-phosphate (S1P) were determined essentially as described in Cinamon et al., Nat Immunol 5:713-720, 2004. Briefly, 5×10⁵ splenocytes were seeded in the upper chamber of transwell plates with a 3 μm filter (Corning Costar Corp.). Transmigration of B-cell subpopulations in response to increasing concentrations of recombinant S1P (Sigma) (0.1-10 nM) was assessed after 3 hours by phenotypic analysis (FACS) of cell populations in the lower chamber.

RNA preparation and real-time PCR. Semi-quantitative PCR was conducted according to standard protocols on a Corbett Rotor-Gene real-time PCR machine (Corbett Research, Sydney, Australia). For each PCR reaction, 1 μl of cDNA was combined with 5 μl SYBR Green JumpStart Taq ReadyMix (Sigma) with 0.5 μl of specific primer (10 mM). All reactions were performed in triplicate. Primers for S1P1, S1P3, and GAPDH are listed as follows: S1P1 forward 5′-GCGCTCAGAGACTTCGTCTT-3′ (SEQ ID NO: 1), reverse 5′-ACCAGCTCACTCGCAAAGTT-3′ (SEQ ID NO: 2); S1P3 forward 5′-CCTTGCAGAACGAGAGCCTA-3′ (SEQ ID NO: 3), reverse 5′-TTCCCGGAGAGTGTCATTTC-3′ (SEQ ID NO: 4); GAPDH forward 5′-CTCATGACCACAGTCCATGC-3′ (SEQ ID NO: 5). All values were normalised to GAPDH mRNA levels.

### 2.2 Results

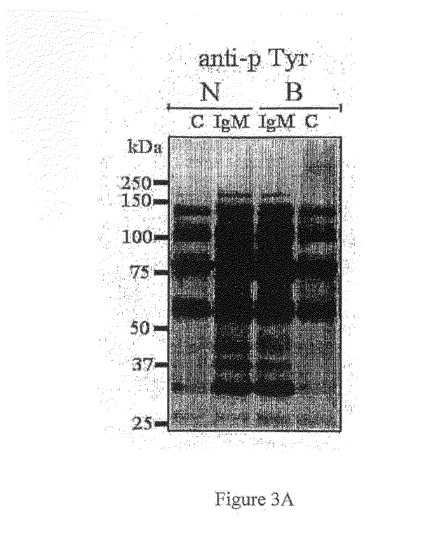

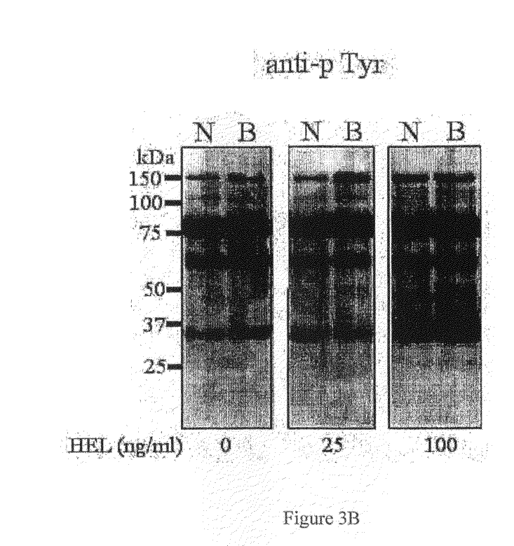

This example investigates the mechanism(s) responsible for the skewed B lymphocyte compartment in NOD mice. In this respect, the level of phosphorylation of signalling proteins in splenic B lymphocytes isolated from wild type C57BL/6 or NOD mice were compared following stimulation with anti-IgM (FIG. 3A). In a second approach, antigen-specific BCR signalling was investigated using in B lymphocytes isolated from C57BL/6 or NOD mice expressing a HEL specific B-cell receptor (BCR) (IgHEL mice) stimulated with their cognate antigen, HEL (FIG. 3B). Regardless of the stimulus, BCR signalling in response to polyclonal or antigen-specific activation was found to be equivalent in both C57BL/6 and NOD mice suggesting that enhanced MZB cell development in NOD mice is not due to altered signalling through the BCR.

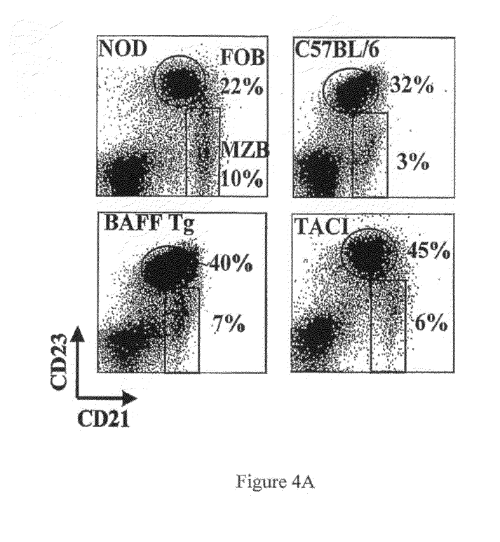

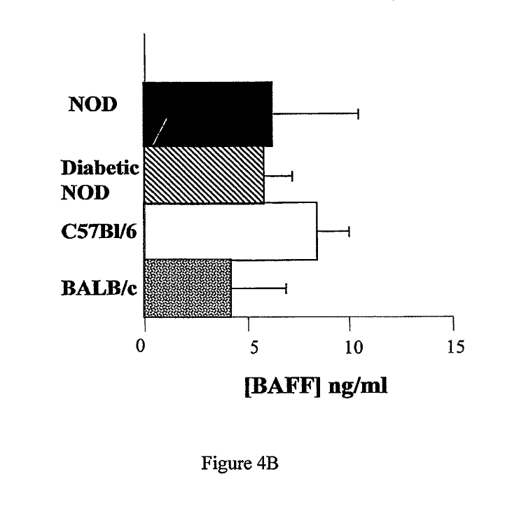

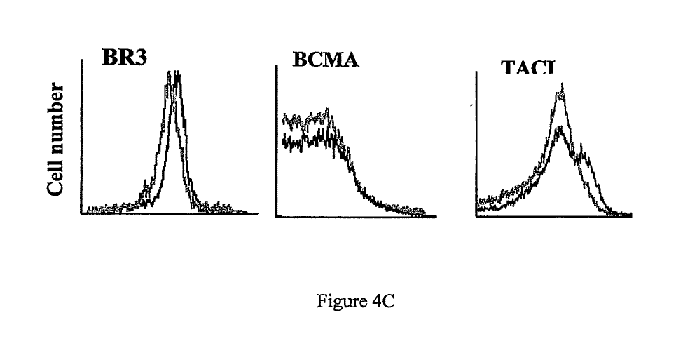

To determine whether or not extrinsic factors might impact upon B lymphocyte development in NOD mice, B-cell-activating-factor-belonging-to-the-TNF-family (BAFF) signalling in NOD mice was assayed. BAFF transgenic mice, as well as mice deficient in the BAFF receptor TACI, have an expanded MZB population (FIG. 4A). Thus dysregulated BAFF signalling can alter B lymphocyte homeostasis to favour MZB cell survival, resulting in their aberrant expansion. Since MZB cells are also expanded in autoimmune prone NOD mice (FIG. 1E), the BAFF system was analyzed to determine whether or not alternations in this system contribute to the MZB cell expansion. Although the serum levels of soluble BAFF in NOD mice were not quantitatively different to that observed for other non-diabetic mouse strains (FIG. 4B), NOD B lymphocyte subsets possessed an increased capacity to bind BAFF. MZB and transitional type 2 (T2) B lymphocytes had demonstrably increased BAFF binding when compared to their C57BL/6 counter parts.

Consistent with higher BAFF binding, the BAFF receptors BAFF-R and TACI, but not BCMA, were shown to be increased on mature NOD B lymphocytes compared to expression levels on B lymphocytes from C57BL/6 mice (FIG. 4C). Thus in NOD mice, increased BAFF binding could provide a survival advantage to MZB cells mediated by increased receptor expression, skewing B lymphocyte development in favour of MZB at the expense of FoB.

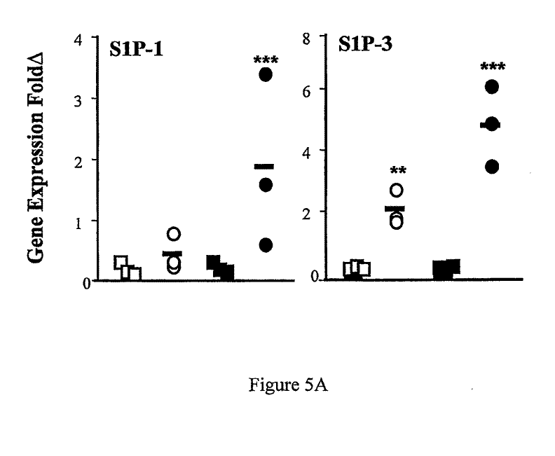

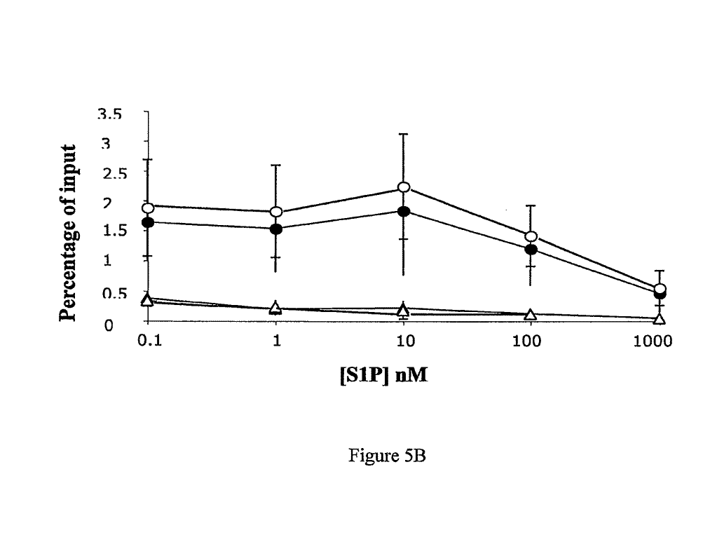

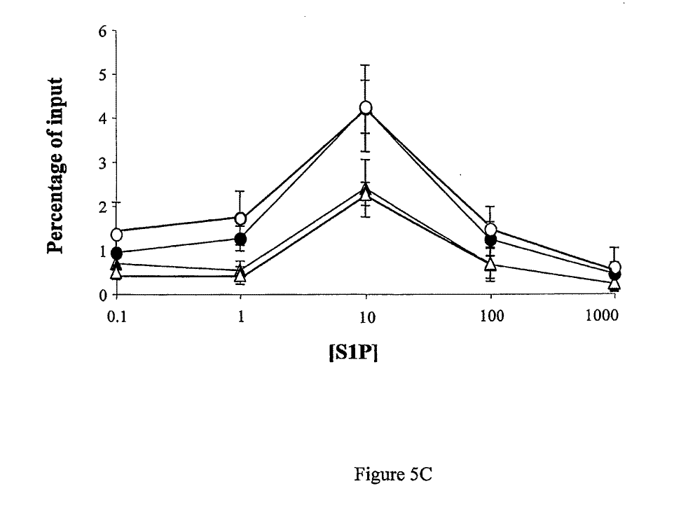

The phospholipid sphingosine-1-phosphate (S1P), and its receptors S1P1 and S1P3 are critical for directing MZB cell localization to the marginal sinus (Cinamon, et al., Nat Immunol 5:713-720, 2004; and Girkontaite, et al., J Exp Med 200:1491-1501, 2004). Moreover, MZB from BAFF transgenic mice express high levels of S1P3, suggestive of a link between MZB expansion, their localisation at the marginal sinus and BAFF. Examination of S1P receptor expression on FoB and MZB cells revealed that S1P1 and S1P3 were more highly expressed on MZB as compared to FoB (FIG. 5A). However, S1P1 and S1P3 receptor expression was significantly increased on MZB from NOD mice compared to MZB from C57BL/6. In contrast, no difference in S1P1 and S1P3 expression on NOD FoB was observed.

To determine the functional significance of increased receptor expression, the in vitro S1P-dependent chemotactic responses of B lymphocytes isolated from C57BL/6 and NOD mice were analyzed. NOD FoB did not exhibit a dose-dependent response to S1P, though their basal migration rate was higher than that observed for FoB from C57BL/6 mice (FIG. 5B). In contrast, and in concordance with increased receptor expression, MZB from NOD mice exhibited a two-fold increase in their chemotactic response to S1P compared with MZB from C57BL/6 mice (FIG. 5C). To determine whether the dynamics of receptor signalling was perturbed in NOD mice, mice were pre-treated with FTY720, an S1P1 agonist which induces receptor internalisation (Cinamon et al., supra). This treatment did not alter the migratory response of NOD FoB or MZB (FIGS. 5B and C), indicating that S1P chemotaxis was dependent upon expression of S1P3, not S1P1 (Cinamon et al., supra). These data indicate that increased sensitivity of MZB to S1P could support their sustained accumulation at the splenic marginal sinus.

### Example 3

### NOD B Cells Exhibit a ‘Hyper’-Active Phenotype

### 3.1 Methods

T-dependent (Ova-specific) and T-independent (Ficoll-specific) immune responses were determined essentially as described in Batten et al., J Immunol 172:812-822, 2004.

CD40 expression was analyzed by standard flow cytometry protocols essentially as described above. Proliferative responses to B cell mitogens were conducted essentially as described in Jin et al., J Immunol 173:657, 2004).

### 3.2 Results

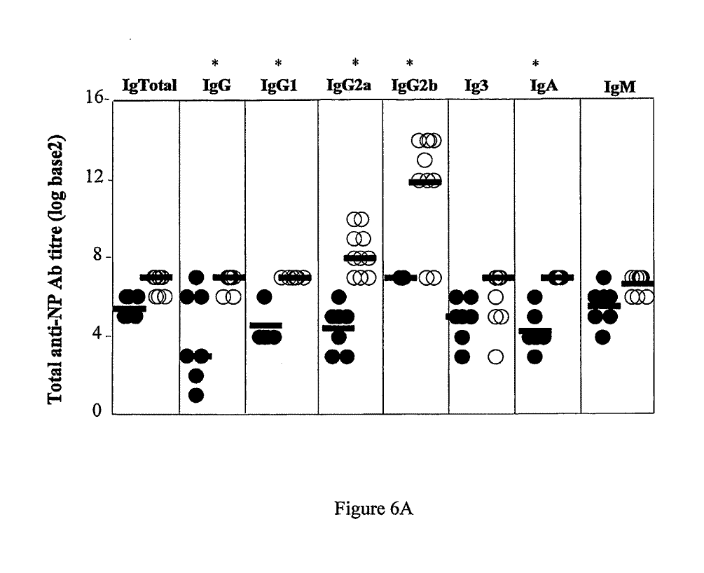

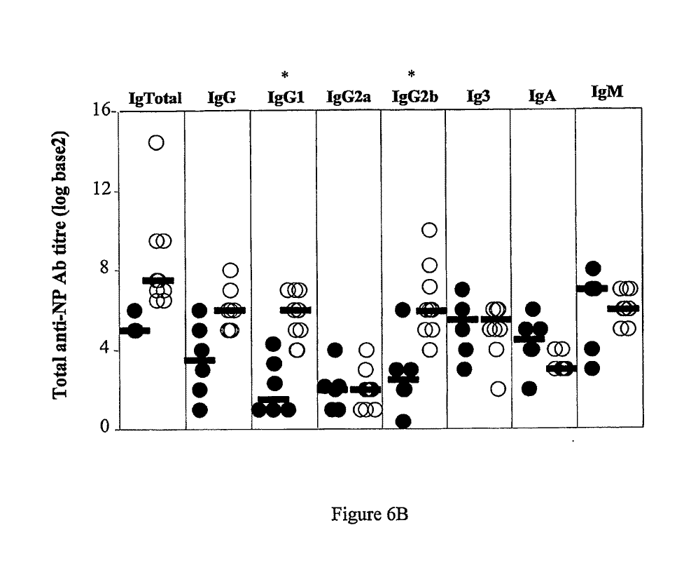

This example studies the antigen responses of the altered mature B lymphocyte subsets in NOD mice. Despite having reduced FoB cell numbers, NOD mice generated exaggerated TD antigen responses (FIG. 6A). High affinity titres for both Th1-type (IgG2a, IgG2b) and Th2-type (IgG1, IgG3, IgA) isotypes were increased, indicating that NOD B cells are generally hyperactive. In addition, examination of TI antigen responses demonstrated that NOD mice have a markedly enhanced IgG₁ and IgG_{2b} isotype response (FIG. 6B). Thus NOD B-lymphocytes are hyper-responsive and exhibit exaggerated antigen-dependent immune-responses, consistent with an increased sensitivity to BAFF and S1P.

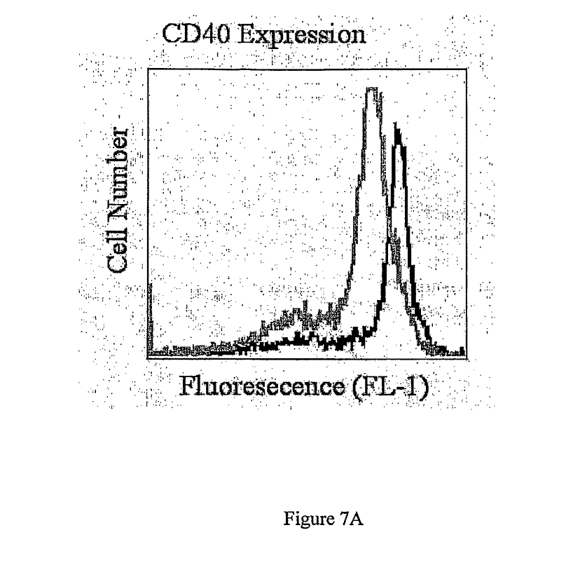

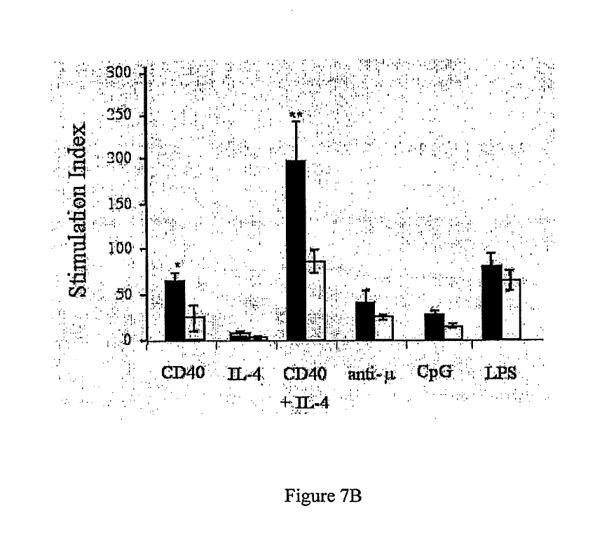

To determine whether or not enhanced expression or signaling via CD40 might contribute to the exaggerated antigen responses of NOD mice, CD40 expression levels on B lymphocytes from NOD mice were analyzed and it was found that they expressed higher levels of CD40 compared to B lymphocytes from C57BL/6 mice (FIG. 7A). To assess the significance of increased CD40 expression, B lymphocyte proliferative responses to CD40 ligation was examined. The proliferative response of NOD B lymphocytes to CD40 ligation, with or without exogenous IL-4, was increased (˜60%) when compared to the proliferative response of B lymphocytes isolated from C57BL/6 mice (FIG. 7B). To evaluate whether NOD B lymphocytes have a generically heightened responsiveness to stimulation, NOD B lymphocyte responses to the B lymphocyte mitogens anti-mμ, CPG, and LPS were examined. As shown in FIG. 7B, there was an equally strong proliferative response to these mitogens in both strains. This was consistent with the normal expression of TLR9 and TLR4 on NOD B lymphocytes. Thus NOD B lymphocytes are hyper-responsive to CD40-ligation and exhibit exaggerated antigen-dependent immune-responses.

These data indicate that the NOD B cell compartment exhibits a hyper-active pro-Th1 type phenotype, regulated in part by enhanced CD40 expression on antibody producing B cells.

### Example 4

### Changes in the B Lymphocyte Compartment During Disease Development

### 4.1 Methods

To analyze the changes in B lymphocyte populations over time and with relation to disease status, NOD mice were aged, fed a standard mouse chow and blood glucose readings were taken weekly to monitor disease onset as previously described (Makhlouf et al., supra; and Grey et al., supra). Lymphocyte suspensions were prepared from spleens of mice at different ages (at least four mice per time point), counted and prepared for phenotypic analysis by flow cytometric analysis using standard protocols as described above.

### 4.2 Results

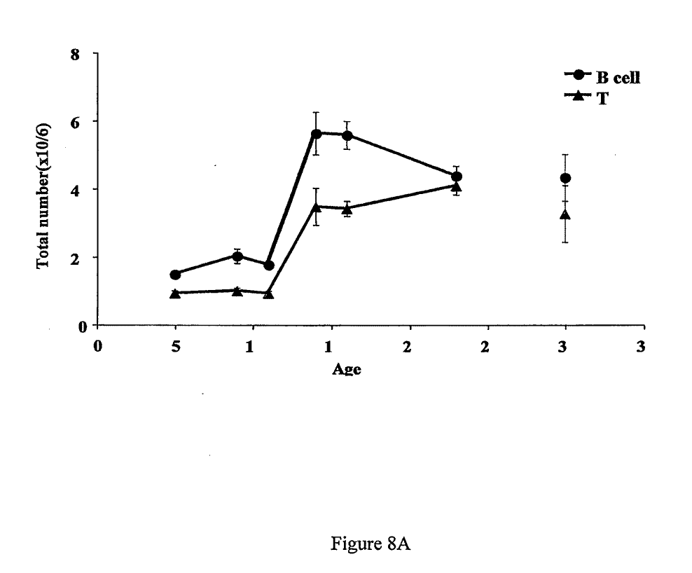

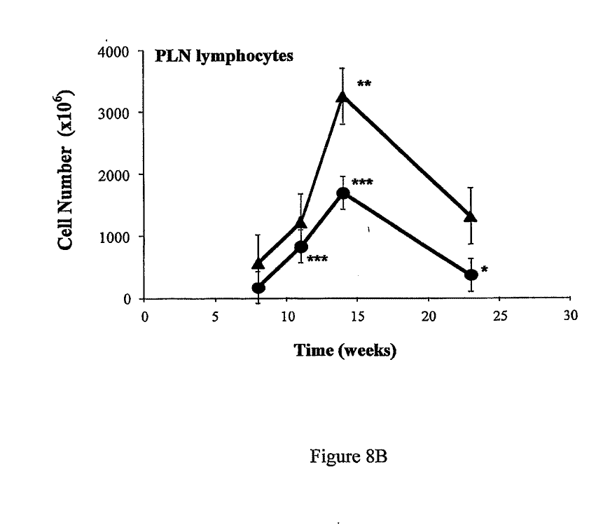

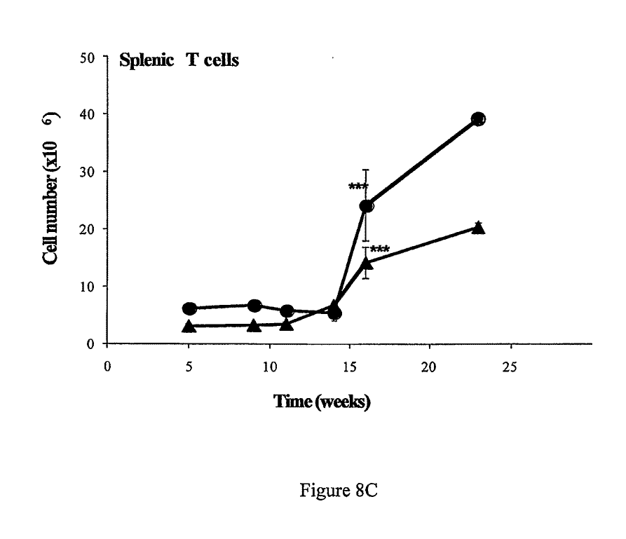

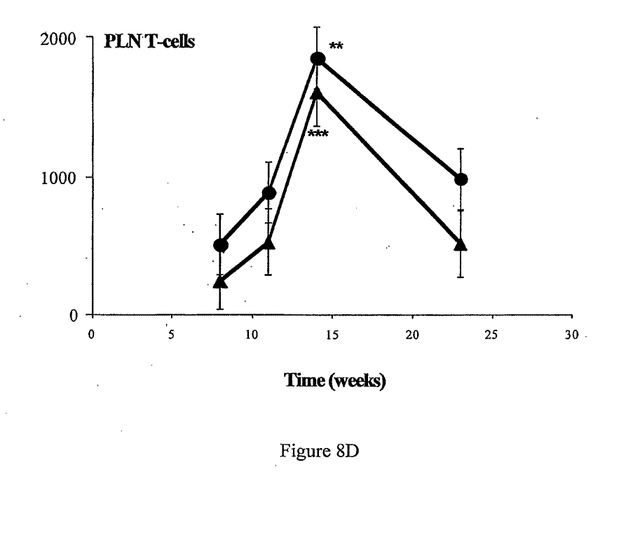

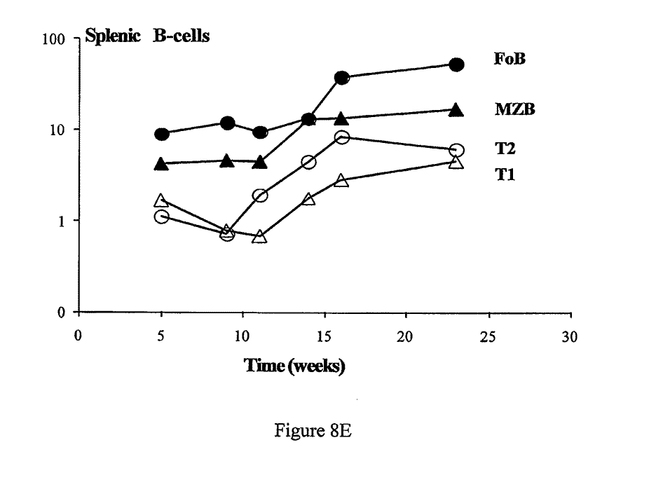

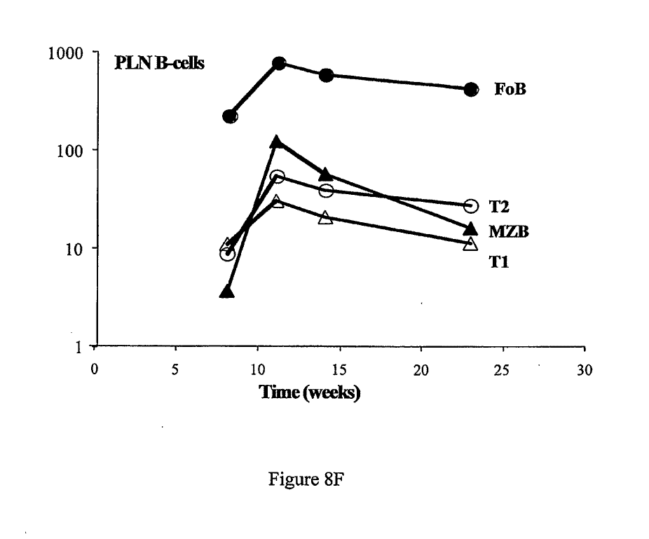

The analysis of the B lymphocyte compartment described supra in young NOD mice revealed a number of marked perturbations that might predispose those mice to disease. This led us to examine how dynamic changes in the peripheral B lymphocyte compartment over time, both in the spleen, blood and the pancreatic lymph nodes (PLN), might relate to disease development. This analysis revealed that the relative lymphopenia in NOD mice was maintained until approximately 11-14 weeks of age, at which point T and B lymphocyte numbers increased dramatically in the spleen and PLN, concurrent with the onset of ‘overt’ diabetes at ˜14 weeks-of-age (FIGS. 8A and B). This increase was not merely developmentally related, as there were no changes in splenocyte numbers in non-diabetic prone mouse strains over this same period (i.e. C57BL/6, DBA, BALB/c; data not shown). The increased cellularity in the spleen and PLN was due to expansion of both CD4⁺ and CD8⁺ T cells (FIGS. 8C and D) as well as B lymphocytes (FIGS. 8E and F). Examination of the B lymphocyte subsets in detail indicated a rise in the absolute numbers of MZB cells and their precursors, T2 cells, at approximately 11 weeks of age in the spleen, occurring prior to the expansion of splenic T- and FoB cells and disease onset, but synchronised with the PLN lymphocyte expansion (FIGS. 8E and F).

### Example 5

### Marginal Zone B Lymphocytes Colonize the NOD Pancreas

### 5.1 Methods

Immunohistochemical analysis using standard protocols described supra, on frozen pancreata isolated from NOD mice at different ages was performed to analyze lymphocytic populations invading the pancreas.

For phenotypic analysis, lymphocyte suspensions were prepared from pancreata and prepared for flow cytometric analysis as described above. At least three mice were examined per time point. Representative data from a single mouse are presented.

### 5.2 Results

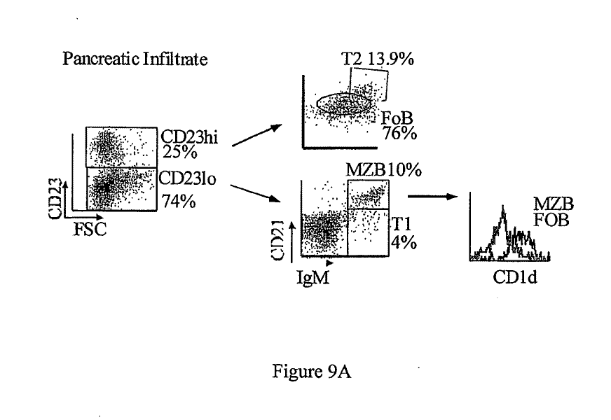

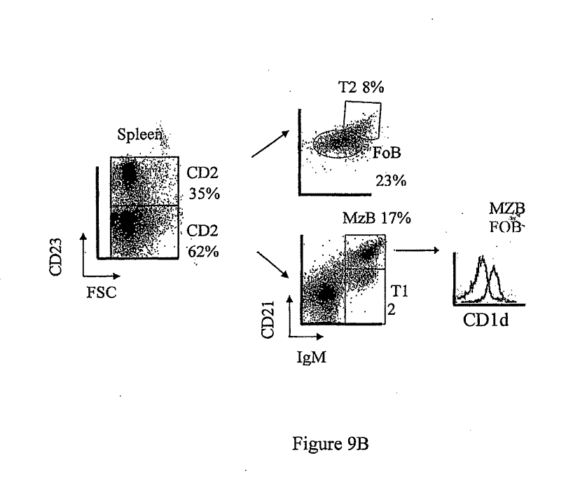

To determine whether or not dysregulated BAFF and S1P signalling might alter B lymphocyte homing to extra lymphoid tissues in NOD mice, the B lymphocyte subsets present in the pancreas of diabetic NOD mice were examined. It was found that B lymphocytes comprised a substantial proportion of the mononuclear cell infiltrate. Subset analysis of the B lymphocytes in the pancreas revealed the presence of a clearly distinguishable FoB (B220⁺, IgM⁺, CD21^{inter}, CD23^{hi}) population (FIG. 9A). A B220⁺, IgM⁺, CD21^{hi}, CD23^{low/-} population reminiscent of splenic MZB cells was also observed in the pancreas of diabetic NOD mice (FIG. 9A). To further characterize those cells, the surface expression of CD1d and CD9 was examined. These markers have been shown to delineate MZB in the spleen (Amano, et al., J Immunol 161:1710-1717, 1998; and Won, et al., J Immunol 168:5605-5611, 2002). These B220⁺, IgM⁺, CD21^{hi}, CD23^{low/-} cells were CD1d^{hi} and CD9⁺, consistent with their identification as MZB. Indeed, these cells expressed a cell surface phenotype identical to that of MZB in the spleen (FIG. 9B).

Subsequently, experiments were performed to whether or not the colonisation of the pancreas by MZB represented a general expansion of this population into extra-splenic compartments by analysing their presence in the blood and peritoneal cavity of NOD mice. Though mature FoB cells were found in these compartments as expected, no MZB were observed in the blood or peritoneal cavity. These data suggest that the colonisation of the pancreas by MZB was antigen specific and related to the pathophysiology of diabetes development.

### Example 6

### Depleting B Cells Protects NOD Mice from Diabetes

### 6.1 Methods

B lymphocyte depletion. To study the effect of eliminating B lymphocytes upon diabetes incidence, NOD mice were treated twice-weekly with 150 μg/ml of BCMA-Fc. BCMA-Fc is a fusion protein consisting of the extra-cellular portion of the BAFF receptor BCMA fused to the Fc domain of human IgG (BCMA-Fc) (Gross, et al., Immunity 15:289-302, 2001). BCMA-Fc was obtained from Biogen Idec, Boston, or produced from a stable-transfectant line expressing BCMA-Fc, cultured in a CELLine bioreactor system (BD Biosciences). The BCMA-Fc fusion protein construct was obtained Dr Pascal Schnieder, University of Lausanne, Switzerland. Mice were treated from 4-6 weeks-of-age, 9-15 weeks-of-age or 12-18 weeks-of-age. Controls were treated with PBS, or 150 μg/ml of intravenous globulin (IVIg).

### 6.2 Results

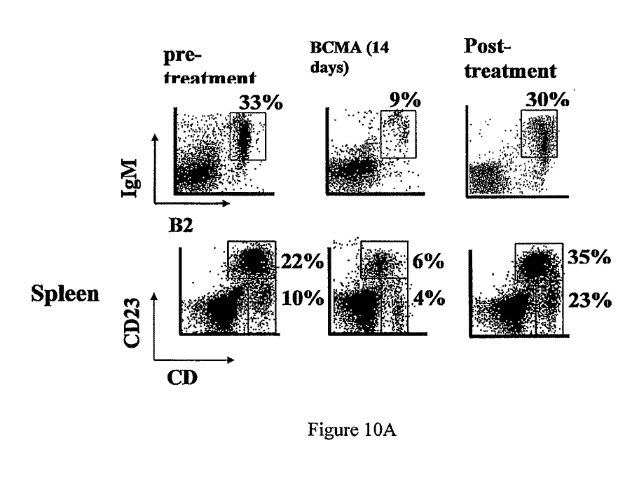

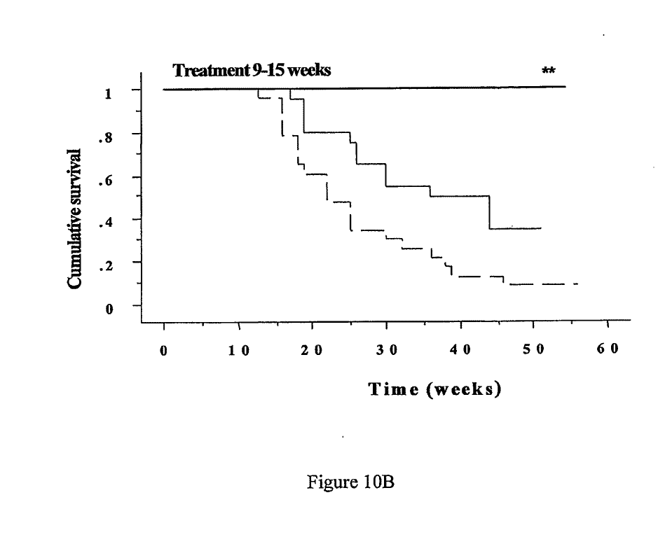

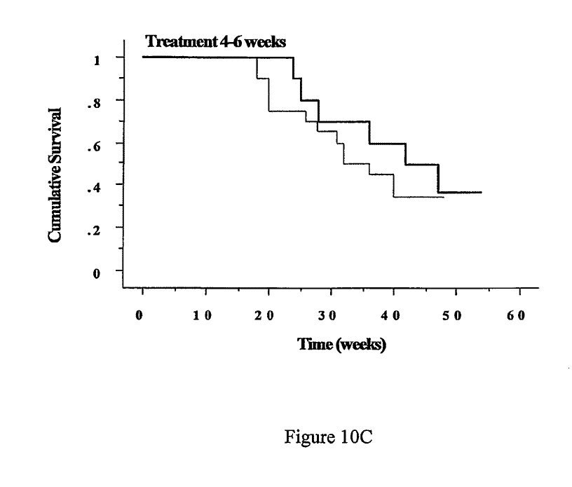

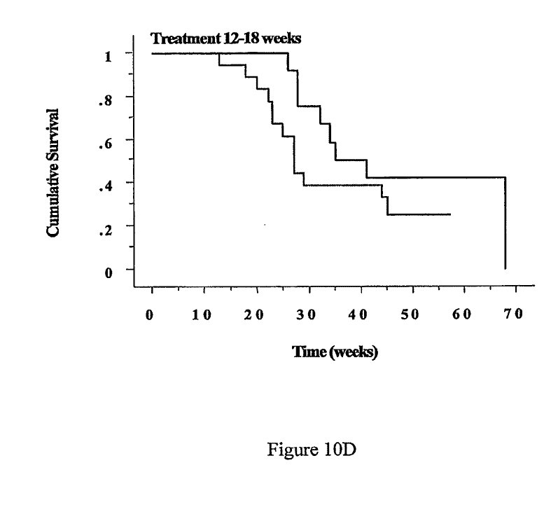

To determine the significance of the staged B lymphocyte expansion between 11-14 weeks-of-age, the effect of temporal elimination of B lymphocytes on disease development was analyzed. B lymphocyte depletion via administration of a soluble BCMA-Fc fusion protein (Pelletier, et al., J Biol Chem 278:33127-33133, 2003). BCMA-Fc binds both BAFF and APRIL leading to a block in B lymphocyte development at the T1-T2 transition (Cinamon, et al., supra). Following initiation of BCMA-Fc treatment, depletion of peripheral B lymphocytes was apparent within about 14 days (FIG. 10A). In contrast peripheral T cell number was unaffected. Once treatment was halted, B lymphocyte numbers returned by about 30 days. Thus to accomplish B lymphocyte depletion at about 11 weeks of age, the point at which B lymphocyte subpopulations began to expand in NOD mice, BCMA-Fc treatment was initiated 14-days earlier at 9 weeks-of-age (FIG. 10B). Control mice were treated with intravenous-immunoglobulin (IVIg) or PBS. NOD mice treated with BCMA-Fc from 9-15 weeks-of-age were completely protected from diabetes. In contrast, control mice developed diabetes with the expected frequency (e.g. ≧70%).

To determine whether the timing of B lymphocyte depletion affected the protective effect of B cell depletion, NOD mice were treated as above except that BCMA-Fc was administered such that B lymphocytes were depleted concomitant with the beginning of insulitis (about 6 weeks) (FIG. 10C), or alternatively, B lymphocytes were depleted just after the expansion of splenic T- and B cells (about 14 weeks) (FIG. 10D).

Despite achieving equivalent B lymphocyte depletion in these experiments, treatment with BCMA-Fc at these times did not protect mice to the same degree as treatment from 9-15 weeks of age.

### Example 7

### BCMA-Fc Treatment Restores Tolerance to Islet Antigens Independent of T-Regulatory Cells

### 7.1 Methods

Adoptive transfer of diabetes. To determine whether protected mice were tolerant of their islets, splenocytes (1×10⁷) from protected mice, treated with BCMA-Fc from 9-15 weeks-of-age, were transferred by intravenous tail vein injection into disease free NOD.SCID recipients. As a control NOD.SCID mice received splenocytes from 8-16 week-old untreated NOD mice with mild hyperglycemia (diabetogenic cells). In some experiments recipients received a 1:1 ratio (2×10⁷ total) of diabetogenic splenocytes mixed with splenocytes from protected mice. Monitoring for hyperglycemia commenced 14 days post transfer.

### 7.2 Results

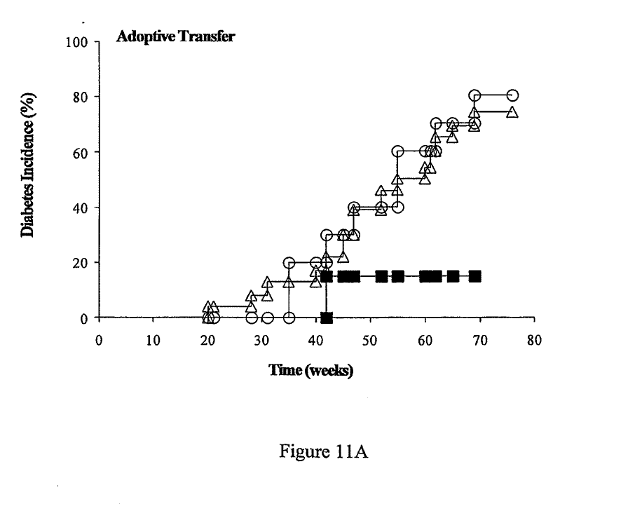

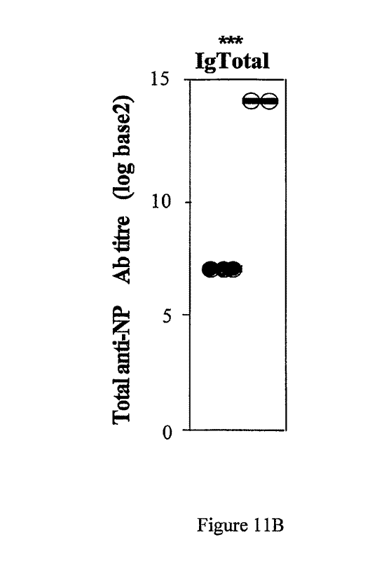

NOD mice were treated with BCMA-Fc from 9-15 weeks-of-age, and at 50 weeks-of-age splenocytes were adoptively transferred from either these disease-free NOD mice, or from newly-diabetic NOD mice (diabetogenic cells) into NOD.SCID (severe combined immune deficient) recipients (FIG. 11A). Approximately 80% of the NOD.SCID mice receiving diabetogenic cells developed diabetes ˜30-60 days post transfer. Whereas the majority of NOD.SCID mice receiving splenocytes from BCMA-Fc treated mice did not develop diabetes.

To assess whether or not tolerance to islet antigens was dominant, co-transfer studies were conducted. However, NOD.SCID recipients receiving a 1:1 ratio of splenocytes from diabetogenic and BCMA-Fc treated mice were not protected from diabetes (FIG. 11A).

The lymphocyte compartment of the BCMA-Fc treated and protected mice was examined at 50 weeks-of-age for alterations that might explain their resistance to diabetes. The ratios of CD4⁺ to CD8⁺ T cells, naïve (CD62L^{hi}, CD44^{lo}) to activated memory/effector (CD62L^{low}, CD44^{hi}) T-cells as well as the T to B lymphocyte ratios were similar to that observed for pre-diabetic and diabetic mice. Further flow cytometric analysis of regulatory cytokine expression by T cells (i.e. γ-IFN, TNF-α, IL-2, IL-10, IL-4), or the presence of CD4⁺ CD25⁺ T-regulatory cells, or NKT cell numbers, did not reveal any obvious changes for these markers and cell populations in the spleen, blood, pancreatic lymph node or pancreas of the protected mice. Together these data indicate that the islet tolerance achieved by BCMA-Fc treatment is not dominant, and is independent of long term T-regulatory cell expansion.

### Example 8

### BCMA-Fc Treated Mice are not Immune Suppressed

### 8.1 Methods

T-dependent (Ova-specific) and T-independent (Ficoll-specific) immune responses were determined as described supra.

### 8.2 Results

To determine that the protected mice were not simply immune suppressed, NOD mice were treated with BCMA-Fc from 9-15 weeks-of-age and at 50 weeks-of-age their T-dependent antigen responses to ovalbumin were analyzed (FIG. 11B). In these protected mice, the total immunoglobulin responses to ovalbumin were elevated compared to younger mice, perhaps as a consequence of the increased B lymphocyte numbers in older mice (FIG. 8). Importantly, this immunisation protocol did not provoke disease, as the BCMA-Fc treated mice maintained normal metabolic control throughout the experimental period. Thus the T and B lymphocyte compartment in BCMA-Fc treated mice was functionally normal and the protected mice were not immune suppressed.

### Example 10

The present studies have identified a distinct period in the pathogenesis of type 1 diabetes, where B lymphocytes are essential for diabetes development. This period is temporally restricted to a period prior to the onset of hyperglycaemia and marked by B lymphocyte hyper-expansion. Depletion of B lymphocytes within this time period prevented diabetes in NOD mice and restored self-tolerance to islet antigens. These studies also demonstrate that B lymphocytes are not required for the initial steps when diabetogenic T cells first encounter autoantigen.

Clinically these studies have important implications. Autoantibodies are predictive of disease onset in human subjects with type 1 diabetes (Tisch and McDevitt Cell 85:291-297, 1996), and these studies show that transient depletion of B lymphocytes within time periods where titres increase may prevent disease. Depletion of B lymphocytes are useful where T cell directed therapies run concomitant risks associated with cytokine release syndrome (Chatenoud et al., Transplantation 49:697-702, 1990) and emergent viral infection (Witherspoon et al., Transplant Sci 4:33-41, 1994).

The use of soluble BAFF antagonists as described herein may offer additional advantages over other B cell treating therapeutics, such as, for example, treatment with anti-CD20 antibodies. Firstly, plasma cells do not express CD20, and, as a consequence, can elude depletion with current antibody-based approaches, whereas BAFF may be required for maintenance of plasmablasts (Avery et al., J Clin Invest 112:286-297, 2003; and O'Connor et al., J Exp Med 199:91-98, 2004). Additionally, BAFF depletion blocks B lymphocyte development at T1-T2 transition (Mackay and Browning Nat Rev Immunol 2:465-475, 2002), allowing more efficient recovery of the B lymphoid compartment following treatment cessation. Finally, transient depletion of B lymphocytes by, for example, BCMA-Fc treatment, restores self-tolerance to islet antigens, to the extent that diabetes does not re-emerge when treatment is stopped and B lymphocyte populations return.

### Example 11

### Detection of Enhanced Levels of T Cells as a Marker of an Immune Response Against Pancreatic β Islet Cells

### 9.1 Methods

Lymphocytes were isolated from spleen, pancreatic lymph nodes (PLN) and pancreas of NOD mice of various ages using standard techniques. Cell suspensions were then labeled with PE-labeled IGRP-tetramer for 1 h, followed by FITC-labeled monoclonal rat antibody against mouse cell surface antigen CD8a (Ly2)(53-6-7) for 30 minutes. Double positive, antigen-specific T cells, were identified by flow cytometry (BD Biosciences) essentially as described in Trudeau et. al, J Clin Invest. 111: 217-23, 2003.

### 9.2 Results

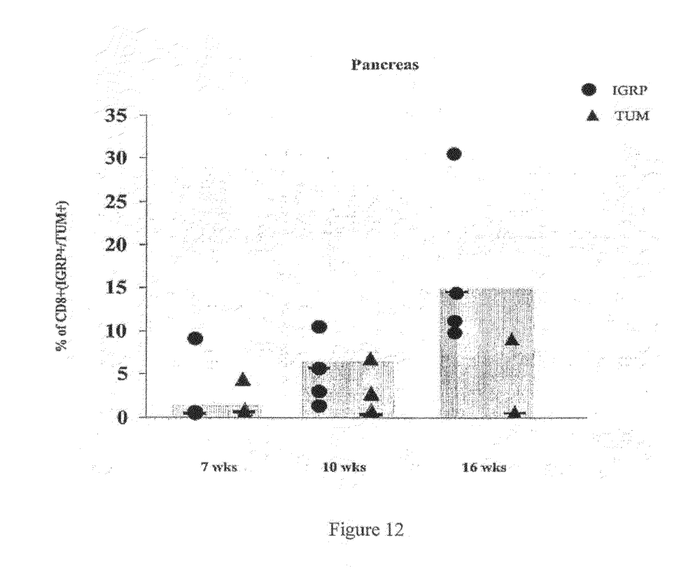

As shown in FIG. 12 the frequency of IGRP+ T cells increases with the onset of diabetes in NOD mice. Flow cytometric analysis of NOD splenocytes demonstrates percentage of CD8+ T cells that specifically recognize the islet auto-antigen IGRP. Accordingly, the detection of IGRP+ T cells is useful for determining the onset of an immune response by a subject against a pancreatic β islet cell, and as a consequence, a suitable time for performing the therapeutic method described herein according to any embodiment.

### Example 12

### Detection of Autoantibodies Against Pancreatic β-Islet Cell Antigens

The following example describes methods for detecting autoantibodies against markers to detect the onset of an immune response against a pancreatic β islet cell.

IAA and insulin antibodies (IA) are measured by binding to ¹²⁵I-labeled insulin in a protein A/G radiobinding assay essentially as previously described for IAA determination in human blood (Naserke, et al., Diabetologia 41: 681-683, 1998) with minor modifications. Mouse serum (2.5 μl) is incubated with 1159 nU human ¹²⁵I-insulin (specific activity 360 μCi/μg; approximately 22,000 cpm; Aventis, Frankfurt, Germany) in 25 μl of 50 mM Tris, 1% Tween 20, pH 8.0, at 4° C. for 72 h before addition of 2 mg of protein A-Sepharose (Pharmacia) and 6 μl of GammaBind Sepharose (Amersham Biosciences, Piscataway, N.J.) suspended in 50 μl of 50 mM Tris, 1% Tween 20, pH 8.0, for 1 h at 4° C. Beads are then washed five times in ice-cold 50 mM Tris, 1% Tween 20, pH 8.0, and counted for 10 min (γ-counter Cobra II, Packard, Meriden, Conn.). Positive standards used in the assay are dilutions of a mouse monoclonal anti-insulin antibody. Results are expressed as an index calculated as (counts per 5 minute [cpm] in the test serum minus cpm of negative serum)/cpm standard minus cpm negative). The upper limit of normal is determined from the 99th centile values obtained in sera from BALB/c and C57/B6 female mice.

Antibodies to glucagon are measured in a protein A/G radiobinding assay. Serum (2.5 μl) is diluted in 25 μl of 50 mM Tris plus 0.1% Tween 20, pH 8.0, and incubated with 25,000 cpm ¹²⁵I-glucagon (Amersham) at 4° C. overnight, before addition of 2 mg of protein A-Sepharose (Pharmacia) and 6 μl of GammaBind Sepharose (Amersham) suspended in 50 μl of 50 mM Tris plus 0.1% Tween 20, pH 8.0, for 1 h at 4° C. Beads are then washed five times in ice-cold 50 mM Tris plus 0.1% Tween 20, pH 8.0, and counted for 10 min (γ-counter Cobra 11; Packard). Positive standards are dilutions of a mouse monoclonal anti-glucagon antibody

GAD and IA-2 antibodies are measured by using radiobinding assays essentially as previously described (Bonifacio, et al., Diabetes 50: 2451-2458, 2001). Serum is incubated overnight at 4° C. with 20,000 cpm of ³⁵S-methionine-labeled in vitro translated GAD or IA-2/IA-2β antigens in 25 μl of 50 mM Tris, 150 mM NaCl, 1% Tween 20, pH 7.4. To each well is added the equivalent of 1 mg of protein A-Sepharose (Pharmacia) and 3 μl of GammaBind Sepharose (Amersham) suspended in 50 μl of 50 mM Tris, 150 mM NaCl, 1% Tween 20, pH 7.4, and plates are incubated for 1 h at 4° C. before washing five times with ice-cold 50 mM Tris, 150 mM NaCl, 1% Tween 20, pH 7.4, and counted (β-Scintillation Counter Top Count; Packard). Results are expressed as cpm. The upper limit of normal is determined from the 99th centile values obtained in sera from BALB/c and C57 Black female mice.

### Example 13

### Treatment of Diabetes

NOD mice are monitored using one or more methods described herein to determine the onset of an immune response against a pancreatic β islet cell.

Following detection of such an immune response, i.e., at about 9-15 weeks-of-age mice are administered on or more of TACI-Ig, BR3-Ig and/or Rituxan.

Suitable dosages are as follows:

| Therapeutic | Dosage |
|---|---|
| TACI-Ig | 20 μg or 100 μg three times per week for 5 weeks |
| BR3-Ig | 100 μg or 200 μg twice weekly for four weeks |
| Anti-CD20 | 100 μg once |
| antibody | |
| (Rituxan) | |

Mice are then assessed for the onset of diabetes as described supra.

We claim:

1. A therapeutic and/or prophylactic method comprising administering to a subject an amount of a composition sufficient to reduce or deplete antibody producing cells and/or prevent expansion of said cells in a tissue or organ of a subject suffering from a T cell mediated autoimmune disease or at risk of suffering from said disease.

2. The method of claim 1 wherein the composition is administered immediately prior to or concomitant with an autoimmune response.

3. The method of claim 2 wherein the autoimmune response is indicated by expansion of a population of T cells and/or B cells and/or by the production of autoantibodies and/or by an increase in serum glucose and/or polyuria and/or polydipsia and/or abnormal β pancreatic islet cell function.

4. The method according to claim 1 wherein the autoimmune disease comprises an autoimmune response against a pancreatic β-islet cell.

5. The method according to claim 1 wherein the autoimmune disease is type 1 diabetes.

6. The method of claim 1, said method comprising administering to a subject an amount of a compound that reduces or depletes antibody producing cells to thereby reduce the number of antibody producing cells and/or prevent expansion of said cells thereby preventing type 1 diabetes or reducing type 1 diabetes disease progression.

7. The method according to claim 6 comprising administering the compound to the subject immediately prior to or concomitantly with the onset of an immune response by the subject against a pancreatic β-islet cell.

8. The method according to claim 7 additionally comprising detecting the onset of the immune response against a pancreatic β-islet cell or predicting the onset of the immune response against a pancreatic β-islet cell prior to administration of the compound.

9. The method according to claim 8 wherein the immune response against a pancreatic β islet cell is indicated by an increase in serum glucose and/or polyuria and/or polydipsia and/or abnormal β pancreatic islet cell function.

10. The method according to claim 8 wherein detecting the onset of the immune response against a pancreatic β-islet cell comprises:
(i) contacting an immunoglobulin containing sample from the subject with a sample comprising pancreatic β cell and/or with a protein expressed by a pancreatic β-cell or an immunogenic fragment or epitope thereof for a time and under conditions sufficient for an antigen-antibody complex to form; and
(ii) detecting the antigen-antibody complex,
wherein detection of the antigen-antibody complex is indicative of the onset of an immune response against a pancreatic β-islet cell by the subject.

11. The method according to claim 8 wherein detecting the onset of the immune response against a pancreatic β-islet cell comprises:
(i) contacting a T cell containing fraction from the subject with a protein expressed by a pancreatic β-cell or an immunogenic fragment or epitope thereof or a protein complex comprising said protein, fragment and/or epitope for a time and under conditions sufficient for a T cell to bind to the protein, fragment, epitope or complex; and
(ii) detecting the T cell bound to the protein, fragment, epitope or complex,
wherein detection of the T cell bound to the protein, fragment, epitope or complex is indicative of the onset of an immune response against a pancreatic β-islet cell by the subject.

12. The method according to claim 11 comprising contacting the T cell fraction with a protein complex comprising an islet-specific glucose-6-phosphatase-related protein and/or an immunogenic fragment and/or epitope thereof.

13. The method according to claim 8 wherein detecting the onset of the immune response against a pancreatic β-islet cell comprises:
(i) determining the number of B cells in a sample from a subject suspected of suffering from or at risk of suffering from type 1 diabetes; and
(ii) comparing the number of B cells determined at (i) to the number of B cells in a reference sample,
wherein an increased number of the B cells or the type of B cell at (i) compared to (ii) is indicative of the onset of an immune response against a pancreatic β-islet cell by the subject.

14. The method according to claim 13 comprising determining the number of marginal-zone (MZ) B cells in the sample from the subject and comparing the number of MZ B cells in the sample from the subject to the number of MZB cells in a reference sample.

15. The method according to claim 13 comprising determining the number of B cells in a whole blood sample or an extract or a fraction thereof.

16. The method according to claim 13 wherein the reference sample is selected from the group consisting of:
(i) a sample from a normal subject;
(ii) a sample from a healthy subject;
(iii) a sample or data set comprising measurements for the subject being tested wherein said sample or measurements have been taken previously, such as, for example, when the subject was known to healthy or, in the case of a subject having the disease, when the subject was diagnosed or at an earlier stage in disease progression;
(iv) an extract of any one of (i) to (iii);
(v) a fraction of any one of (i) to (iii);
(vi) a data set comprising measurements of the number of B cells in a sample from a healthy individual or a population of normal individuals;
(vii) a data set comprising measurements of the number of B cells in a sample from a normal individual or a population of normal individuals

17. The method according to claim 16 wherein the reference sample is (i) or (ii).

18. The method according to claim 1 wherein the composition binds to a protein expressed on the surface of a B cell and prevents B cell development and/or kills the B cell.

19. The method according to claim 18 wherein the composition comprises an antibody and/or a humanized antibody and/or a chimeric antibody and/or a recombinant antibody and/or an antibody fragment that binds to CD20.

20. The method according to claim 1 wherein the composition binds to a protein required for B cell development and/or B cell survival to thereby reduce the number of antibody producing cells in the subject.

21. The method according to claim 20 wherein the composition binds to a B-cell-activating-factor-belonging-to-the-TNF-family (BAFF) polypeptide to thereby reduce the number of antibody producing cells in the subject.

22. The method according to claim 21 wherein the composition is a fusion protein comprising an extracellular domain of a BAFF-receptor and a Fc domain of human immunoglobulin G.

23. The method according to claim 22 wherein the composition comprises a compound selected from the group consisting of BCMA-Ig, TACI-Ig and BR3-Ig and mixtures thereof.

24. The method according to claim 1 additionally comprising ceasing administering the composition to the subject following administration of the composition for a time sufficient to reduce the number of antibody producing cells in the subject.

25. The method according to claim 1 additionally comprising determining the number of antibody producing cells in the subject following administration of the composition and ceasing administering the composition if the number of antibody producing cells is sufficiently reduced for effective treatment.

26. A method for preventing type 1 diabetes onset in a subject in need thereof, said method comprising administering to the subject an amount of a fusion protein comprising an extracellular domain of a B-cell maturation antigen (BCMA) polypeptide and a Fc domain of human immunoglobulin G (Ig) to thereby reduce the number of antibody producing cells and/or prevent expansion of said cells, wherein said compound is administered immediately prior to or concomitant with the onset of an autoimmune response against a pancreatic β-islet cell as determined by expansion of cytotoxic T cells against pancreatic β-islet cells or pancreatic β-islet cell markers and/or autoantibodies against one or more pancreatic β-islet cell markers, thereby preventing type 1 diabetes onset.

27. A use of a composition sufficient to reduce or deplete antibody producing cells and/or prevent expansion of said cells in the manufacture of a medicament for the treatment and/or prevention of T cell mediated autoimmune disease.

28. The use according to claim 27 wherein the medicament for administration immediately prior to or concomitant with an autoimmune response.

29. The use according to claim 27 wherein the autoimmune disease is type 1 diabetes.

30. The use according to claim 27 wherein the composition comprises a compound selected from the group consisting of BCMA-Ig, TACI-Ig and BR3-Ig and mixtures thereof.

31. The use according to claim 27 wherein the composition comprises an antibody and/or a humanized antibody and/or a chimeric antibody and/or a recombinant antibody and/or an antibody fragment that binds to CD20.

32. A composition sufficient to reduce or deplete antibody producing cells and/or prevent expansion of said cells for use in the treatment and/or prevention of T cell mediated autoimmune disease.

33. The composition according to claim 32 when used to treat and/or prevent T cell mediated autoimmune disease.

34. The composition according to claim 32 when administered to a subject suffering from or at risk of suffering from T cell mediated autoimmune disease.

35. The composition according to claim 32 for administration immediately prior to or concomitant with an autoimmune response.

36. The composition according to claim 32 wherein the autoimmune disease is type 1 diabetes.

37. The composition according to claim 32 comprising a compound selected from the group consisting of BCMA-Ig, TACI-Ig and BR3-Ig and mixtures thereof.

38. The composition according to claim 32 comprising an antibody and/or a humanized antibody and/or a chimeric antibody and/or a recombinant antibody and/or an antibody fragment that binds to CD20.
